(19) United States
(12) Patent Application Publication
(10) Pub. No.: US 2026/0318864 A1
(43) Pub. Date: Oct. 15, 2026

(54) GAS SENSOR AND CONTROL METHOD OF GAS SENSOR

(71) Applicant: NGK INSULATORS, LTD., Nagoya-shi (JP)

(72) Inventors: Junya SUGIURA, Nagoya-shi (JP); Yusuke WATANABE, Nagoya-shi (JP); Shingo TANAKA, Kasugai-shi (JP)

(21) Appl. No.: 19/466,649

(22) Filed: Feb. 2, 2026

(30) Foreign Application Priority Data
Feb. 10, 2025 (JP) 2025-019828

(51) Int. Cl. G01N 27/41; G01N 27/409; G01N 33/00

(52) U.S. Cl. CPC G01N 27/41; G01N 27/409; G01N 33/004

(57) ABSTRACT

A gas sensor includes a sensor element and a control unit. The sensor element includes: an oxygen pump cell; a first measurement pump cell; and a second measurement pump cell. The control unit comprises: a pump control part and a concentration calculating part. The pump control part has an oxygen measurement mode and a normal measurement mode; and the concentration calculating part calculates an oxygen concentration in the oxygen measurement mode; calculates a water vapor concentration in the measurement-object gas based on a current flowing through the first measurement pump cell in the normal measurement mode and the oxygen concentration in the oxygen measurement mode; and calculates a carbon dioxide concentration in the measurement-object gas based on a current flowing through the second measurement pump cell in the normal measurement mode and the oxygen concentration in the oxygen measurement mode.

### CROSS-REFERENCE TO RELATED APPLICATION

The present application claims priority from Japanese Patent Application No. 2025-019828, filed on Feb. 10, 2025, the content of which is hereby incorporated by reference into this application.

### BACKGROUND OF THE INVENTION

### Technical Field of the Invention

The present invention relates to a gas sensor for detecting water vapor and carbon dioxide in a measurement-object gas.

### Background Art

Measurement of concentration of an objective gas component (oxygen O₂, water vapor H₂O, carbon dioxide CO₂, nitrogen oxide NOx, ammonia NH₃, hydrocarbon HC, etc.) in a measurement-object gas is required in various fields, for example, in combustion control and exhaust gas control of an internal combustion engine such as an engine of automobile, in environment control, in a medical field, a biotechnology field, and an agriculture and industry field. Various measurement devices are used for the concentration measurement, and as an example, a limiting current-type gas sensor using an oxygen ion conductive solid electrolyte such as zirconia (ZrO₂) is known.

For example, JP 5918177 B2 discloses a gas sensor that identifies concentrations of a water vapor component and a carbon dioxide component of a measurement gas based on a current flowing through a solid electrolyte. For example, WO 2024/202731 A1 discloses a gas sensor that measures a carbon dioxide concentration in a measurement-object gas, and WO 2024/202763 A1 discloses a gas sensor that measures a water concentration in a measurement-object gas. Further, for example, JP 2023-152599 A discloses a gas sensor capable of measuring a water vapor concentration, a carbon dioxide concentration, and an oxygen concentration in a measurement-object gas.

### CITATION LIST

Patent Documents
Patent Document 1: JP 5918177 B2
Patent Document 2: WO 2024/202731 A1
Patent Document 3: WO 2024/202763 A1
Patent Document 4: JP 2023-152599 A

### SUMMARY OF THE INVENTION

### Problems to be solved by the Invention

Oxygen and carbon dioxide and/or water vapor generally coexist in a measurement-object gas. Specifically, an exhaust gas from an internal combustion engine such as an engine of automobile contains a plurality of components such as oxygen, water vapor, carbon dioxide, hydrocarbon gas, and noninflammable gas, and a component ratio of the components and a gas temperature may momently change. Accordingly, the measurement-object gas is generally a gas containing a plurality of gas species.

The gas sensor is required to accurately measure a concentration of a target gas to be measured regardless of gas composition in a measurement-object gas. For example, WO 2024/202731 A1 discloses a gas sensor that determines a carbon dioxide concentration by taking a water concentration in account. Further, for example, WO 2024/202763 A1 discloses a gas sensor that determines a water concentration by taking a carbon dioxide concentration in account.

When concentration of water vapor and/or carbon dioxide in a measurement-object gas is a target gas to be measured, a gas sensor is required to accurately measure the water vapor concentration and/or the carbon dioxide concentration in the measurement-object gas regardless of a concentration of a gas (for example, oxygen) other than the target gas to be measured in the measurement-object gas.

It is therefore an object of the present invention to provide a gas sensor that can accurately measure a water vapor concentration and a carbon dioxide concentration in a measurement-object gas regardless of an oxygen concentration in the measurement-object gas.

### Means for solving the problems

As a result of intensive studies, the present inventors have found that, by performing an oxygen measurement mode and a normal measurement mode, and considering an oxygen concentration in the oxygen measurement mode in calculation of a water vapor concentration and a carbon dioxide concentration, it is possible to accurately measure the water vapor concentration and the carbon dioxide concentration in the measurement-object gas regardless of the oxygen concentration in the measurement-object gas. The present invention includes the following aspects.

(1) A gas sensor for detecting water vapor and carbon dioxide in a measurement-object gas, the gas sensor comprising a sensor element and a control unit for controlling the sensor element, wherein
the sensor element comprises:
a base part in an elongated plate shape, including an oxygen-ion-conductive solid electrolyte layer;
a measurement-object gas flow cavity having a gas inlet that opens on a surface of the base part, a first internal cavity that communicates with the gas inlet via a first diffusion-rate limiting path, and a second internal cavity that communicates with the first internal cavity via a second diffusion-rate limiting path;
an oxygen pump cell including: an intracavity oxygen pump electrode disposed in the first internal cavity of the measurement-object gas flow cavity; and an extracavity oxygen pump electrode disposed at a position different from the measurement-object gas flow cavity on the base part and corresponding to the intracavity oxygen pump electrode;
a first measurement pump cell including: an intracavity first measurement electrode disposed in the second internal cavity of the measurement-object gas flow cavity; and an extracavity first measurement electrode disposed at a position different from the measurement-object gas flow cavity on the base part and corresponding to the intracavity first measurement electrode;
a second measurement pump cell including: an intracavity second measurement electrode disposed at a position opposite to the second diffusion-rate limiting path with respect to the intracavity first measurement electrode in the second internal cavity, or in a third internal cavity that communicates with the second internal cavity via a third diffusion-rate limiting path of the measurement-object gas flow cavity; and an extracavity second measurement electrode disposed at a position different from the measurement-object gas flow cavity on the base part and corresponding to the intracavity second measurement electrode;
a reference gas chamber formed inside the base part, and being separated from the measurement-object gas flow cavity; and
a reference electrode disposed in the reference gas chamber; and
the control unit comprises:
a pump control part for controlling operation of the oxygen pump cell, the first measurement pump cell and the second measurement pump cell, and
a concentration calculating part for calculating a concentration of each of water vapor and carbon dioxide in a measurement-object gas, wherein
the pump control part has an oxygen measurement mode and a normal measurement mode;
in the oxygen measurement mode, the pump control part controls the operation of the oxygen pump cell, and
in the normal measurement mode,
the pump control part applies a predetermined oxygen pump voltage between the intracavity oxygen pump electrode and the extracavity oxygen pump electrode of the oxygen pump cell to pump out oxygen from the first internal cavity and decompose the water vapor and the carbon dioxide in the measurement-object gas in the first internal cavity;
the pump control part applies a predetermined first measurement pump voltage between the intracavity first measurement electrode and the extracavity first measurement electrode of the first measurement pump cell to pump oxygen into the second internal cavity and selectively oxidize, in the second internal cavity, hydrogen generated by decomposition of the water vapor; and
the pump control part applies a predetermined second measurement pump voltage between the intracavity second measurement electrode and the extracavity second measurement electrode of the second measurement pump cell to pump oxygen into near a surface of the intracavity second measurement electrode and selectively oxidize, near the surface of the intracavity second measurement electrode, carbon monoxide generated by decomposition of the carbon dioxide; and
the concentration calculating part calculates an oxygen concentration in the oxygen measurement mode;
the concentration calculating part calculates a water vapor concentration in the measurement-object gas based on a current flowing through the first measurement pump cell in the normal measurement mode and the oxygen concentration in the oxygen measurement mode; and
the concentration calculating part calculates a carbon dioxide concentration in the measurement-object gas based on a current flowing through the second measurement pump cell in the normal measurement mode and the oxygen concentration in the oxygen measurement mode.

(2) The gas sensor according to the above (1), wherein
in the oxygen measurement mode, the pump control part applies a predetermined oxygen pump voltage at which neither the water vapor nor the carbon dioxide is decomposed on the intracavity oxygen pump electrode between the intracavity oxygen pump electrode and the extracavity oxygen pump electrode to pump out oxygen from the first internal cavity; and
the concentration calculating part calculates the oxygen concentration in the oxygen measurement mode based on a current flowing though the oxygen pump cell in the oxygen measurement mode.

(3) The gas sensor according to the above (2), wherein
in the oxygen measurement mode, the pump control part applies the predetermined oxygen pump voltage at which neither the water vapor nor the carbon dioxide is decomposed on the intracavity oxygen pump electrode between the intracavity oxygen pump electrode and the extracavity oxygen pump electrode based on a voltage between the intracavity oxygen pump electrode and the reference electrode to pump out oxygen from the first internal cavity.

(4) The gas sensor according to the above (2) or (3), wherein the predetermined oxygen pump voltage applied between the intracavity oxygen pump electrode and the extracavity oxygen pump electrode in the oxygen measurement mode is lower than the predetermined oxygen pump voltage applied between the intracavity oxygen pump electrode and the extracavity oxygen pump electrode in the normal measurement mode.

(5) The gas sensor according to the above (3), wherein the voltage between the intracavity oxygen pump electrode and the reference electrode is controlled at 200 mV or more and 700 mV or less in the oxygen measurement mode.

(6) The gas sensor according to the above (1), wherein
the sensor element further comprises a diffusion resister arranged around the extracavity oxygen pump electrode;
in the oxygen measurement mode, the pump control part applies a predetermined oxygen pump voltage at which neither the water vapor nor the carbon dioxide is decomposed on the extracavity oxygen pump electrode between the intracavity oxygen pump electrode and the extracavity oxygen pump electrode to pump oxygen into the first internal cavity; and
the concentration calculating part calculates the oxygen concentration in the oxygen measurement mode based on a current flowing though the oxygen pump cell in the oxygen measurement mode.

(7) The gas sensor according to the above (6), wherein
in the oxygen measurement mode, the pump control part applies the predetermined oxygen pump voltage at which neither the water vapor nor the carbon dioxide is decomposed on the extracavity oxygen pump electrode between the intracavity oxygen pump electrode and the extracavity oxygen pump electrode based on a voltage between the intracavity oxygen pump electrode and the reference electrode to pump oxygen into the first internal cavity.

(8) The gas sensor according to the above (7), wherein the voltage between the intracavity oxygen pump electrode and the reference electrode is controlled at -700 mV or more and -200 mV or less in the oxygen measurement mode.

(9) The gas sensor according to the above (6), wherein
in the oxygen measurement mode, the pump control part applies the predetermined oxygen pump voltage at which neither the water vapor nor the carbon dioxide is decomposed on the extracavity oxygen pump electrode between the intracavity oxygen pump electrode and the extracavity oxygen pump electrode based on a voltage between the extracavity oxygen pump electrode and the reference electrode to pump oxygen into the first internal cavity.

(10) The gas sensor according to the above (9), wherein the voltage between the extracavity oxygen pump electrode and the reference electrode is controlled at 200 mV or more and 700 mV or less in the oxygen measurement mode.

(11) The gas sensor according to any one of the above (6) to (10), wherein an absolute value of the predetermined oxygen pump voltage applied between the intracavity oxygen pump electrode and the extracavity oxygen pump electrode in the oxygen measurement mode is lower than an absolute value of the predetermined oxygen pump voltage applied between the intracavity oxygen pump electrode and the extracavity oxygen pump electrode in the normal measurement mode.

(12) The gas sensor according to any one of the above (6) to (11), wherein the diffusion resister is a porous layer covering the extracavity oxygen pump electrode.

(13) The gas sensor according to any one of the above (1) to (12), wherein
the concentration calculating part corrects the current flowing through the first measurement pump cell in the normal measurement mode based on the oxygen concentration in the oxygen measurement mode to calculate a corrected current, and calculates the water vapor concentration in the measurement-object gas based on the corrected current; and
the concentration calculating part corrects the current flowing through the second measurement pump cell in the normal measurement mode based on the oxygen concentration in the oxygen measurement mode to calculate a corrected current, and calculates the carbon dioxide concentration in the measurement-object gas based on the corrected current.

(14) The gas sensor according to any one of the above (1) to (13), whereinin the normal measurement mode,
the pump control part applies the predetermined oxygen pump voltage between the intracavity oxygen pump electrode and the extracavity oxygen pump electrode of the oxygen pump cell based on a voltage between the intracavity oxygen pump electrode and the reference electrode to pump out oxygen from the first internal cavity and decompose the water vapor and the carbon dioxide in the measurement-object gas in the first internal cavity;
the pump control part applies the predetermined first measurement pump voltage between the intracavity first measurement electrode and the extracavity first measurement electrode of the first measurement pump cell based on a voltage between the intracavity first measurement electrode and the reference electrode to pump oxygen into the second internal cavity and selectively oxidize, in the second internal cavity, the hydrogen generated by the decomposition of the water vapor; and
the pump control part applies the predetermined second measurement pump voltage between the intracavity second measurement electrode and the extracavity second measurement electrode of the second measurement pump cell based on a voltage between the intracavity second measurement electrode and the reference electrode to pump oxygen into near the surface of the intracavity second measurement electrode and selectively oxidize, near the surface of the intracavity second measurement electrode, the carbon monoxide generated by the decomposition of the carbon dioxide.

(15) The gas sensor according to any one of the above (1) to (14), wherein the pump control part repeatedly performs the oxygen measurement mode and the normal measurement mode.

(16) The gas sensor according to any one of the above (1) to (15), wherein
a duration time of the oxygen measurement mode is 0.1 second or more and 3 seconds or less, and
a duration time of the normal measurement mode is 1 second or more and 20 seconds or less.

(17) A control method of a gas sensor for detecting water vapor and carbon dioxide in a measurement-object gas, the gas sensor being the gas sensor according to any one of the above (1) to (16), and
the control method comprising:
an oxygen concentration measuring step of calculating an oxygen concentration in the oxygen measurement mode;
a water vapor concentration measuring step of calculating a water vapor concentration in the measurement-object gas based on a current flowing through the first measurement pump cell in the normal measurement mode and the oxygen concentration in the oxygen measurement mode just before the normal measurement mode; and
a carbon dioxide concentration measuring step of calculating a carbon dioxide concentration in the measurement-object gas based on a current flowing through the second measurement pump cell in the normal measurement mode and the oxygen concentration in the oxygen measurement mode just before the normal measurement mode.

### Advantageous Effect of the Invention

According to the present invention, it is possible to provide a gas sensor that can accurately measure a water vapor concentration and a carbon dioxide concentration in a measurement-object gas regardless of an oxygen concentration in the measurement-object gas.

### BRIEF DESCRIPTION OF THE DRAWINGS

FIG. 1 is a vertical sectional schematic view in a longitudinal direction of a sensor element 101, showing one example of a schematic configuration of a gas sensor 100.

FIG. 2 is a block diagram showing electric connections between a control unit 90 and respective pump cells 21, 50 and 41, and respective sensor cells 80, 81, 82, and 83 of the sensor element 101.

FIG. 3 is a graph showing one example of a relationship between oxygen concentration (O₂ concentration) in a measurement-object gas and a pump current Ip1. The horizontal axis of the graph represents the O₂ concentration [%] and the vertical axis of the graph represents the pump current Ip1 [μA].

FIG. 4 is a graph showing one example of a relationship between oxygen concentration (O₂ concentration) in a measurement-object gas and a pump current Ip2. The horizontal axis of the graph represents the O₂ concentration [%] and the vertical axis of the graph represents the pump current Ip2 [μA].

FIG. 5 is a graph showing one example of a relationship between oxygen concentration (O₂ concentration) in the measurement-object gas and a correction value with respect to the pump current Ip1. The horizontal axis of the graph represents the O₂ concentration [%] and the vertical axis of the graph represents the correction value with respect to the pump current Ip1 [μA].

FIG. 6 is a graph showing one example of a relationship between oxygen concentration (O₂ concentration) in the measurement-object gas and a correction value with respect to the pump current Ip2. The horizontal axis of the graph represents the O₂ concentration [%] and the vertical axis of the graph represents the correction value with respect to the pump current Ip2 [μA].

FIG. 7 is a flow chart of concentration calculating processing of water vapor H₂O and carbon dioxide CO₂ in a measurement-object gas.

FIG. 8 is a vertical sectional schematic view in a longitudinal direction of a sensor element 201, showing one example of a general configuration of the gas sensor 200 including the sensor element 201.

FIG. 9 is a vertical sectional schematic view in a longitudinal direction of a sensor element 301, showing one example of a schematic configuration of a gas sensor 300 including the sensor element 301.

### MODES FOR CARRYING OUT OF THE INVENTION

A gas sensor of the present invention is a gas sensor that detects water vapor and carbon dioxide in a measurement-object gas, and includes a sensor element and a control unit for controlling the sensor element.

The sensor element contained in the gas sensor of the present invention includes:
a base part in an elongated plate shape, including an oxygen-ion-conductive solid electrolyte layer;
a measurement-object gas flow cavity having a gas inlet that opens on a surface of the base part, a first internal cavity that communicates with the gas inlet via a first diffusion-rate limiting path, and a second internal cavity that communicates with the first internal cavity via a second diffusion-rate limiting path;
an oxygen pump cell including: an intracavity oxygen pump electrode disposed in the first internal cavity of the measurement-object gas flow cavity; and an extracavity oxygen pump electrode disposed at a position different from the measurement-object gas flow cavity on the base part and corresponding to the intracavity oxygen pump electrode;
a first measurement pump cell including: an intracavity first measurement electrode disposed in the second internal cavity of the measurement-object gas flow cavity; and an extracavity first measurement electrode disposed at a position different from the measurement-object gas flow cavity on the base part and corresponding to the intracavity first measurement electrode;
a second measurement pump cell including: an intracavity second measurement electrode disposed at a position opposite to the second diffusion-rate limiting path with respect to the intracavity first measurement electrode in the second internal cavity, or in a third internal cavity that communicates with the second internal cavity via a third diffusion-rate limiting path of the measurement-object gas flow cavity; and an extracavity second measurement electrode disposed at a position different from the measurement-object gas flow cavity on the base part and corresponding to the intracavity second measurement electrode;
a reference gas chamber formed inside the base part, and being separated from the measurement-object gas flow cavity; and
a reference electrode disposed in the reference gas chamber.

The sensor element contained in the gas sensor of the present invention may be configured such that the measurement-object gas flow cavity has a gas inlet that opens on a surface of the base part, a first internal cavity that communicates with the gas inlet via a first diffusion-rate limiting path, a second internal cavity that communicates with the first internal cavity via a second diffusion-rate limiting path, and a third internal cavity that communicates with the second internal cavity via a third diffusion-rate limiting path; and
the second measurement pump cell includes: an intracavity second measurement electrode disposed in the third internal cavity of the measurement-object gas flow cavity; and an extracavity second measurement electrode disposed at a position different from the measurement-object gas flow cavity on the base part and corresponding to the intracavity second measurement electrode.

The sensor element contained in the gas sensor of the present invention may be configured such that the measurement-object gas flow cavity has a gas inlet that opens on a surface of the base part, a first internal cavity that communicates with the gas inlet via a first diffusion-rate limiting path, and a second internal cavity that communicates with the first internal cavity via a second diffusion-rate limiting path; and
the second measurement pump cell includes: an intracavity second measurement electrode disposed at a position opposite to the second diffusion-rate limiting path with respect to the intracavity first measurement electrode in the second internal cavity of the measurement-object gas flow cavity; and an extracavity second measurement electrode disposed at a position different from the measurement-object gas flow cavity on the base part and corresponding to the intracavity second measurement electrode.

The control unit contained in the gas sensor of the present invention includes:
a pump control part for controlling operation of the oxygen pump cell, the first measurement pump cell and the second measurement pump cell, and
a concentration calculating part for calculating a concentration of each of water vapor and carbon dioxide in a measurement-object gas, wherein
the pump control part has an oxygen measurement mode and a normal measurement mode;
in the oxygen measurement mode, the pump control part controls the operation of the oxygen pump cell, and
in the normal measurement mode,
the pump control part applies a predetermined oxygen pump voltage between the intracavity oxygen pump electrode and the extracavity oxygen pump electrode of the oxygen pump cell to pump out oxygen from the first internal cavity and decompose the water vapor and the carbon dioxide in the measurement-object gas in the first internal cavity;
the pump control part applies a predetermined first measurement pump voltage between the intracavity first measurement electrode and the extracavity first measurement electrode of the first measurement pump cell to pump oxygen into the second internal cavity and selectively oxidize, in the second internal cavity, hydrogen generated by decomposition of the water vapor; and
the pump control part applies a predetermined second measurement pump voltage between the intracavity second measurement electrode and the extracavity second measurement electrode of the second measurement pump cell to pump oxygen into near a surface of the intracavity second measurement electrode and selectively oxidize, near the surface of the intracavity second measurement electrode, carbon monoxide generated by decomposition of the carbon dioxide; and
the concentration calculating part calculates an oxygen concentration in the oxygen measurement mode;
the concentration calculating part calculates a water vapor concentration in the measurement-object gas based on a current flowing through the first measurement pump cell in the normal measurement mode and the oxygen concentration in the oxygen measurement mode; and
the concentration calculating part calculates a carbon dioxide concentration in the measurement-object gas based on a current flowing through the second measurement pump cell in the normal measurement mode and the oxygen concentration in the oxygen measurement mode.

### Schematic configuration of gas sensor

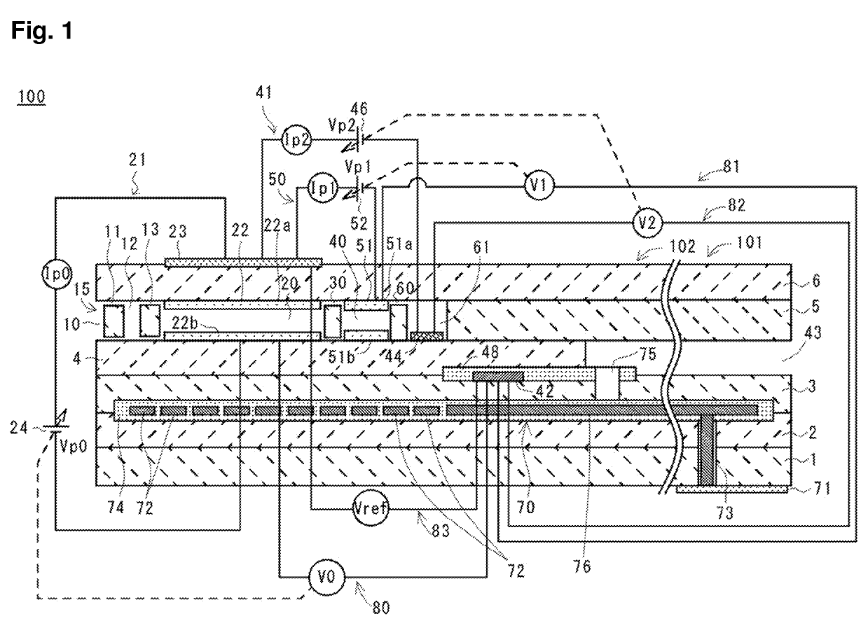

The gas sensor of the present invention will now be described with reference to the drawings. FIG. 1 is a vertical sectional schematic view in a longitudinal direction of a sensor element 101, showing one example of a schematic configuration of a gas sensor 100 including the sensor element 101. Hereinafter, based on FIG. 1, the upper side and the lower side in FIG. 1 are respectively defined as top and bottom, and the left side and the right side in FIG. 1 are respectively defined as a front end side and a rear end side.

In the embodiment of FIG. 1, the gas sensor 100 represents one example of a limiting current-type gas sensor that detects water vapor and carbon dioxide in a measurement-object gas by the sensor element 101, and measures the concentrations of the water vapor and the carbon dioxide.

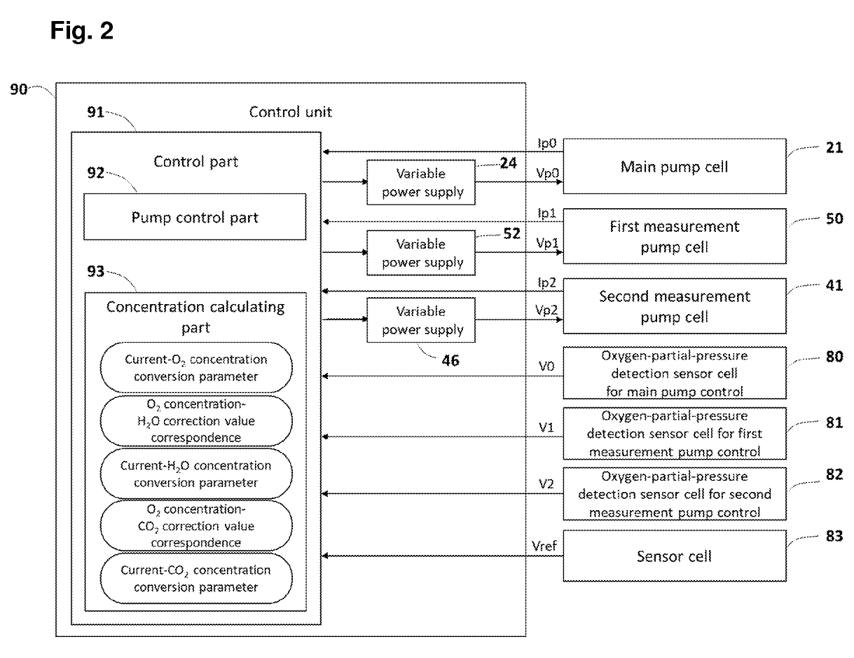

Further, the gas sensor 100 includes a control unit 90 for controlling the sensor element 101. FIG. 2 is a block diagram showing electric connections between the control unit 90 and the sensor element 101.

### Sensor element

The sensor element 101 is an element in an elongated plate shape, including a base part 102 having such a structure that a plurality of oxygen-ion-conductive solid electrolyte layers are layered. The elongated plate shape also called a long plate shape or a belt shape. The base part 102 has such a structure that six layers, namely, a first substrate layer 1, a second substrate layer 2, a third substrate layer 3, a first solid electrolyte layer 4, a spacer layer 5, and a second solid electrolyte layer 6, are layered in this order from the bottom side, as viewed in the drawing. Each of the six layers is formed of an oxygen-ion-conductive solid electrolyte layer containing, for example, zirconia (ZrO₂). The solid electrolyte forming these six layers is dense and gastight. These six layers all may have the same thickness, or the thickness may vary among the layers. The layers are adhered to each other with an adhesive layer of a solid electrolyte interposed therebetween, and the base part 102 includes the adhesive layer. While a layer configuration composed of the six layers is illustrated in FIG. 1, the layer configuration in the present invention is not limited to this, and any number of layers and any layer configuration are possible.

A measurement-object gas flow cavity 15 has a gas inlet 10 that opens on a surface of the base part 102, a first internal cavity 20 that communicates with the gas inlet 10 via a first diffusion-rate limiting path 11 (namely, a first diffusion-rate limiting part), and a second internal cavity 40 that communicates with the first internal cavity 20 via a second diffusion-rate limiting path 30 (namely, a second diffusion-rate limiting part). In this embodiment, the measurement-object gas flow cavity 15 further has a third internal cavity 61 that communicates with the second internal cavity 40 via a third diffusion-rate limiting path 60 (namely, a third diffusion-rate limiting part). That is, this embodiment is an example of a structure having three internal cavities, namely, the first internal cavity 20 that communicates with the gas inlet 10 via the first diffusion-rate limiting path 11, the second internal cavity 40 that communicates with the first internal cavity 20 via the second diffusion-rate limiting path 30, and the third internal cavity 61 that communicates with the second internal cavity 40 via the third diffusion-rate limiting path 60.

The gas inlet 10 is formed between the lower surface of the second solid electrolyte layer 6 and the upper surface of the first solid electrolyte layer 4 in one end part in the longitudinal direction (hereinafter, referred to as a front end part) of the sensor element 101. The measurement-object gas flow cavity 15, that is, a measurement-object gas flow part is formed in such a form that the first diffusion-rate limiting path 11, a buffer space 12, a fourth diffusion-rate limiting path 13 (namely, a fourth diffusion-rate limiting part), the first internal cavity 20, the second diffusion-rate limiting path 30, the second internal cavity 40, the third diffusion-rate limiting path 60, and the third internal cavity 61 communicate in this order in the longitudinal direction from the gas inlet 10.

The gas inlet 10, the buffer space 12, the first internal cavity 20, the second internal cavity 40, and the third internal cavity 61 constitute internal spaces of the sensor element 101. Each of the internal spaces is provided in such a manner that a portion of the spacer layer 5 is hollowed out, and the top of each of the internal spaces is defined by the lower surface of the second solid electrolyte layer 6, the bottom of each of the internal spaces is defined by the upper surface of the first solid electrolyte layer 4, and the lateral surface of each of the internal spaces is defined by the lateral surface of the spacer layer 5.

Each of the first diffusion-rate limiting path 11, the fourth diffusion-rate limiting path 13, and the second diffusion-rate limiting path 30 is provided as two laterally elongated slits (having the longitudinal direction of the openings in the direction perpendicular to the figure in FIG. 1). Each of the first diffusion-rate limiting path 11, the fourth diffusion-rate limiting path 13, and the second diffusion-rate limiting path 30 may be in such a form that a desired diffusion resistance is created, but the form is not limited to the slits.

The third diffusion-rate limiting path 60 is provided as a single laterally elongated slit (having the longitudinal direction of the opening in the direction perpendicular to the figure in FIG. 1) between the spacer layer 5 and the second solid electrolyte layer 6. The third diffusion-rate limiting path 60 may be in such a form that a desired diffusion resistance is created, but the form is not limited to the slit.

Also, at a position farther from the front end than the measurement-object gas flow cavity 15, a reference gas introduction space 43 is disposed between the upper surface of the third substrate layer 3 and the lower surface of the spacer layer 5 at a position where the reference gas introduction space 43 is laterally defined by the lateral surface of the first solid electrolyte layer 4. The reference gas introduction space 43 has an opening in the other end part (hereinafter, referred to as a rear end part) of the sensor element 101. As a reference gas for concentration measurement, for example, air is introduced into the reference gas introduction space 43.

An air introduction layer 48 is a layer formed of porous alumina, and is so configured that a reference gas is introduced into the air introduction layer 48 via the reference gas introduction space 43. The air introduction layer 48 is formed to cover a reference electrode 42. In this embodiment, the air introduction layer 48 and the reference gas introduction space 43 correspond to the reference gas chamber of the present invention.

The reference electrode 42 is an electrode disposed in the reference gas chamber. The reference electrode 42 is an electrode sandwiched between the upper surface of the third substrate layer 3 and the first solid electrolyte layer 4, and as described above, the air introduction layer 48 leading to the reference gas introduction space 43 is disposed around the reference electrode 42. That is, the reference electrode 42 is disposed to be in contact with a reference gas via the air introduction layer 48 which is a porous material, and the reference gas introduction space 43. As will be described later, the reference electrode 42 can be used to measure the oxygen concentration (oxygen partial pressure) in the first internal cavity 20, the second internal cavity 40, and the third internal cavity 61. The reference electrode 42 may be formed as a porous cermet electrode (e.g., a cermet electrode of Pt and ZrO₂) in a rectangular shape in a planar view.

In the measurement-object gas flow cavity 15, the gas inlet 10 is open to the external space, and the measurement-object gas is taken into the sensor element 101 from the external space through the gas inlet 10.

In the present embodiment, the measurement-object gas flow cavity 15 is in such a form that the measurement-object gas is introduced through the gas inlet 10 that is open on the front end surface of the sensor element 101, however, the present invention is not limited to this form. For example, the measurement-object gas flow cavity 15 need not have a recess of the gas inlet 10. In this case, the first diffusion-rate limiting path 11 serves as both a gas inlet and a first diffusion-rate limiting path.

For example, the measurement-object gas flow cavity 15 may have an opening that communicates with the buffer space 12 or a position near the buffer space 12 of the first internal cavity 20, on a lateral surface along the longitudinal direction of the base part 102. In this case, the measurement-object gas is introduced from the lateral surface along the longitudinal direction of the base part 102 through the opening.

Further, for example, the measurement-object gas flow cavity 15 may be so configured that the measurement-object gas is introduced through a porous body.

The first diffusion-rate limiting path 11 creates a predetermined diffusion resistance to the measurement-object gas taken through the gas inlet 10.

The buffer space 12 is provided to mitigate the influence of pressure fluctuation on the detected value when the pressure of the measurement-object gas fluctuates. The sensor element 101 may have a structure without the buffer space 12.

The fourth diffusion-rate limiting path 13 creates a predetermined diffusion resistance to the measurement-object gas introduced into the first internal cavity 20 from the buffer space 12. The fourth diffusion-rate limiting path 13 is provided in association with the buffer space 12 being provided.

When the buffer space 12 and the fourth diffusion-rate limiting path 13 are not provided, the first diffusion-rate limiting path 11 directly communicates with the first internal cavity 20.

The first internal cavity 20 is provided as a space for adjusting the oxygen partial pressure in the measurement-object gas introduced through the fourth diffusion-rate limiting path 13. The oxygen partial pressure is adjusted by operation of a main pump cell 21. That is, the main pump cell 21 functions as an oxygen pump cell of the present invention.

The main pump cell 21 is an electrochemical pump cell including the inner main pump electrode 22 as an intra-cavity oxygen pump electrode disposed in the first internal cavity 20 of the measurement-object gas flow cavity 15, and an outer pump electrode 23 as an extra-cavity oxygen pump electrode disposed at a position different from the measurement-object gas flow cavity 15 on the base part 102 (in FIG. 1, on an outer surface of the base part 102) and corresponding to the inner main pump electrode 22. The phrase “corresponding to the inner main pump electrode 22” means that the outer pump electrode 23 and the inner main pump electrode 22 are provided with the second solid electrolyte layer 6 being interposed therebetween.

That is, the main pump cell 21 is an electrochemical pump cell composed of the inner main pump electrode 22 having a ceiling electrode portion 22a disposed over substantially the entire surface of the lower surface of the second solid electrolyte layer 6 that faces the first internal cavity 20, the outer pump electrode 23 disposed on the upper surface of the second solid electrolyte layer 6 so as to be exposed to the external space, and the second solid electrolyte layer 6 sandwiched between the inner main pump electrode 22 and the outer pump electrode 23.

The inner main pump electrode 22 is formed to span the upper and lower solid electrolyte layers (the second solid electrolyte layer 6 and the first solid electrolyte layer 4) that define the first internal cavity 20 and the spacer layer 5 that defines the lateral wall. Specifically, the ceiling electrode portion 22a is formed on the lower surface of the second solid electrolyte layer 6 that defines the ceiling surface of the first internal cavity 20, and a bottom electrode portion 22b is formed on the upper surface of the first solid electrolyte layer 4 that defines the bottom surface of the first internal cavity 20. Also, lateral electrode portions (not shown) are formed on the lateral wall surfaces (inner surface) of the spacer layer 5 that form both lateral wall parts of the first internal cavity 20 so as to connect the ceiling electrode portion 22a and the bottom electrode portion 22b. Thus, the inner main pump electrode 22 is provided as a tunnel-like structure in the area where the lateral electrode portions are disposed.

The inner main pump electrode 22 and the outer pump electrode 23 are porous cermet electrodes (electrodes in a state that a metal component and a ceramic component are mixed) in a rectangular shape in a planar view. The inner main pump electrode 22 and the outer pump electrode 23 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, each of the inner main pump electrode 22 and the outer pump electrode 23 may be a porous cermet electrode made of Pt and ZrO₂.

In the main pump cell 21, a pump voltage (oxygen pump voltage) Vp0 is applied between the inner main pump electrode 22 and the outer pump electrode 23 by a variable power supply 24 to make a pump current Ip0 flow between the inner main pump electrode 22 and the outer pump electrode 23 in either a positive or negative direction, and thus it is possible to pump out oxygen in the first internal cavity 20 to the external space or pump oxygen into the first internal cavity 20 from the external space.

The inner main pump electrode 22, the second solid electrolyte layer 6, the spacer layer 5, the first solid electrolyte layer 4, the third substrate layer 3, and the reference electrode 42 form an electrochemical sensor cell, namely, an oxygen-partial-pressure detection sensor cell 80 for main pump control. In the oxygen-partial-pressure detection sensor cell 80 for main pump control, an electromotive force (a voltage V0) is generated between the inner main pump electrode 22 and the reference electrode 42 due to a difference in oxygen concentration between an atmosphere in the first internal cavity 20 and a reference gas in the reference gas introduction space 43.

The oxygen concentration (oxygen partial pressure) in the first internal cavity 20 can be detected from the voltage V0 measured in the oxygen-partial-pressure detection sensor cell 80 for main pump control. In addition, the pump current Ip0 is controlled by performing feedback control of the pump voltage Vp0 in the variable power supply 24 so that the voltage V0 is constant. Thus, the oxygen partial pressure in the first internal cavity 20 can be maintained at a predetermined value.

The second diffusion-rate limiting path 30 creates a predetermined diffusion resistance to the measurement-object gas whose oxygen concentration (oxygen partial pressure) has been controlled in the first internal cavity 20 by the operation of the main pump cell 21, and guides the measurement-object gas into the second internal cavity 40.

The second internal cavity 40 is provided as a space for performing processing related to measurement of a water vapor concentration in the measurement-object gas introduced through the second diffusion-rate limiting path 30. By operation of a first measurement pump cell 50, oxygen is supplied from the outside of the sensor element 101 to the second internal cavity 40.

The first measurement pump cell 50 is an electrochemical pump cell including a first measurement electrode 51 as an intracavity first measurement electrode disposed in the second internal cavity 40 of the measurement-object gas flow cavity 15, and an extracavity first measurement electrode disposed at a position different from the measurement-object gas flow cavity 15 on the base part 102 and corresponding to the first measurement electrode 51. In this embodiment, the outer pump electrode 23 disposed on the outer surface of the base part 102 functions also as the extracavity first measurement electrode. The phrase “corresponding to the first measurement electrode 51” means that the outer pump electrode 23 and the first measurement electrode 51 are provided with the second solid electrolyte layer 6 being interposed therebetween.

That is, the first measurement pump cell 50 is an electrochemical pump cell composed of the first measurement electrode 51 having a ceiling electrode portion 51a disposed on substantially the entire surface of the lower surface of the second solid electrolyte layer 6 facing with the second internal cavity 40, the outer pump electrode 23 (the outer electrode is not limited to the outer pump electrode 23, but may be any suitable electrode disposed at a position different from the measurement-object gas flow cavity 15, for example, outside the sensor element 101), and the second solid electrolyte layer 6.

The first measurement electrode 51 is disposed in the second internal cavity 40 in a tunnel-like structure similar to the inner main pump electrode 22 disposed in the first internal cavity 20. Specifically, in the tunnel-like structure, the ceiling electrode portion 51a is formed on the second solid electrolyte layer 6 that defines the ceiling surface of the second internal cavity 40, a bottom electrode portion 51b is formed on the first solid electrolyte layer 4 that defines the bottom surface of the second internal cavity 40, and lateral electrode portions (not shown) connecting the ceiling electrode portion 51a and the bottom electrode portion 51b are formed on the wall surfaces of the spacer layer 5 that define the lateral walls of the second internal cavity 40.

The first measurement electrode 51 is a porous cermet electrode in a rectangular shape in a planar view, as with the case of the inner main pump electrode 22 and the outer pump electrode 23. The first measurement electrode 51 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, the first measurement electrode 51 may be a porous cermet electrode made of Pt containing 0.1 wt.% to 30.0 wt.% of Au and ZrO₂.

In the first measurement pump cell 50, by applying a pump voltage (first measurement pump voltage) Vp1 between the first measurement electrode 51 and the outer pump electrode 23 by a variable power supply 52 to make a pump current (water vapor detecting current) Ip1 flow between the first measurement electrode 51 and the outer pump electrode 23, it is possible to pump oxygen into the second internal cavity 40. In FIG. 1, a direction of pumping oxygen into the second internal cavity 40 is illustrated as a negative direction.

The first measurement electrode 51, the reference electrode 42, the second solid electrolyte layer 6, the spacer layer 5, the first solid electrolyte layer 4, and the third substrate layer 3 form an electrochemical sensor cell, namely, an oxygen-partial-pressure detection sensor cell 81 for first measurement pump control. In the oxygen-partial-pressure detection sensor cell 81 for first measurement pump control, an electromotive force (a voltage V1) is generated between the first measurement electrode 51 and the reference electrode 42 due to a difference in oxygen concentration between an atmosphere in the second internal cavity 40 and a reference gas in the reference gas introduction space 43.

The oxygen concentration (oxygen partial pressure) in the second internal cavity 40 can be detected from the voltage V1 measured in the oxygen-partial-pressure detection sensor cell 81 for first measurement pump control. The pump voltage Vp1 in the variable power supply 52 is feedback controlled on the basis of the voltage V1 detected by the oxygen-partial-pressure detection sensor cell 81 for first measurement pump control.

The third diffusion-rate limiting path 60 creates a predetermined diffusion resistance to the measurement-object gas whose oxygen concentration (oxygen partial pressure) has been controlled in the second internal cavity 40 by the operation of the first measurement pump cell 50, and guides the measurement-object gas into the third internal cavity 61.

The third internal cavity 61 is provided as a space for performing processing related to measurement of a carbon dioxide concentration in the measurement-object gas introduced through the third diffusion-rate limiting path 60. By operation of a second measurement pump cell 41, oxygen is supplied from the outside of the sensor element 101 to the third internal cavity 61.

The second measurement pump cell 41 is an electrochemical pump cell including a second measurement electrode 44 as an intracavity second measurement electrode disposed in the third internal cavity 61 of the measurement-object gas flow cavity 15, and an extracavity second measurement electrode disposed at a position different from the measurement-object gas flow cavity 15 on the base part 102 and corresponding to the second measurement electrode 44. In this embodiment, the outer pump electrode 23 disposed on the outer surface of the base part 102 functions also as the extracavity second measurement electrode. The phrase “corresponding to the second measurement electrode 44” means that the outer pump electrode 23 and the second measurement electrode 44 are provided with the second solid electrolyte layer 6, the spacer layer 5, and the first solid electrolyte layer 4 being interposed therebetween.

That is, the second measurement pump cell 41 is an electrochemical pump cell composed of the second measurement electrode 44 disposed on an upper surface of the first solid electrolyte layer 4 facing with the third internal cavity 61, the outer pump electrode 23 (the outer electrode is not limited to the outer pump electrode 23, but may be any suitable electrode disposed at a position different from the measurement-object gas flow cavity 15, for example, outside the sensor element 101), the second solid electrolyte layer 6, the spacer layer 5, and the first solid electrolyte layer 4. The second measurement electrode 44 is disposed at a position opposite to the second diffusion-rate limiting path 30 with respect to the first measurement electrode 51, that is, at a position farther from the second diffusion-rate limiting path 30 than the first measurement electrode 51.

The second measurement electrode 44 is a porous cermet electrode in a rectangular shape in a planar view, as with the case of the inner main pump electrode 22 and the outer pump electrode 23. The second measurement electrode 44 may contain a noble metal having catalytic activity (e.g., at least one of Pt, Rh, Ir, Ru, and Pd) as the metal component. The ceramic component to be used is not particularly limited, but is preferably an oxygen-ion-conductive solid electrolyte as in the case of the base part 102. For example, the second measurement electrode 44 may be a porous cermet electrode made of Pt and ZrO₂.

In the second measurement pump cell 41, by applying a pump voltage (second measurement pump voltage) Vp2 between the second measurement electrode 44 and the outer pump electrode 23 by a variable power supply 46 to make a pump current (carbon dioxide detecting current) Ip2 flow between the second measurement electrode 44 and the outer pump electrode 23, it is possible to pump oxygen into the third internal cavity 61. In FIG. 1, a direction of pumping oxygen into the third internal cavity 61 is illustrated as a negative direction.

To detect the oxygen partial pressure around the second measurement electrode 44, the first solid electrolyte layer 4, the third substrate layer 3, the second measurement electrode 44, and the reference electrode 42 constitute an electrochemical sensor cell, namely an oxygen-partial-pressure detection sensor cell 82 for second measurement pump control. In the oxygen-partial-pressure detection sensor cell 82 for second measurement pump control, an electromotive force (a voltage V2) is generated between the second measurement electrode 44 and the reference electrode 42 due to a difference in oxygen concentration between an atmosphere in the third internal cavity 61 and a reference gas in the reference gas introduction space 43.

The oxygen concentration (oxygen partial pressure) in the third internal cavity 61 can be detected from the voltage V2 measured in the oxygen-partial-pressure detection sensor cell 82 for second measurement pump control. The pump voltage Vp2 in the variable power supply 46 is feedback controlled on the basis of the voltage V2 detected by the oxygen-partial-pressure detection sensor cell 82 for second measurement pump control.

Also, the second solid electrolyte layer 6, the spacer layer 5, the first solid electrolyte layer 4, the third substrate layer 3, the outer pump electrode 23, and the reference electrode 42 constitute an electrochemical sensor cell 83, and it is possible to detect the oxygen partial pressure in the measurement-object gas outside the sensor by an electromotive force (a voltage Vref) obtained by the sensor cell 83. Thus, a function as an oxygen concentration cell may be added to the gas sensor 100.

The sensor element 101 further includes a heater part 70 that functions as a temperature regulator of heating and maintaining the temperature of the sensor element 101 so as to enhance the oxygen ion conductivity of the solid electrolyte. The heater part 70 includes a heater electrode 71, a heater 72, a heater lead 76, a through hole 73, a heater insulating layer 74, and a pressure relief vent 75.

The heater electrode 71 is an electrode formed in contact with the lower surface of the first substrate layer 1. The power can be supplied to the heater part 70 from the outside by connecting the heater electrode 71 with an external power supply.

The heater 72 is an electrical resistor sandwiched by the second substrate layer 2 and the third substrate layer 3 from top and bottom. The heater 72 is connected with the heater electrode 71 via the heater lead 76 that connects with the heater 72 and extends in the rear end side in the longitudinal direction of the sensor element 101, and the through hole 73. The heater 72 is externally powered through the heater electrode 71 to generate heat, and heats and maintains the temperature of the solid electrolyte forming the sensor element 101.

The heater 72 is embedded over the whole area from the first internal cavity 20 to the third internal cavity 61 so that the temperature of the sensor element 101 can be adjusted to such a temperature that activates the solid electrolyte. The temperature may be adjusted so that the main pump cell 21, the first measurement pump cell 50, and the second measurement pump cell 41 are operable. It is not necessary that the whole area is adjusted to the same temperature, but the sensor element 101 may have temperature distribution. The sensor element 101 (base part 102) may have temperature distribution in the longitudinal direction, and may have temperature distribution in a thickness direction and/or a width direction. For example, the sensor element 101 may be heated so that temperature of the solid electrolyte around the measurement-object gas flow cavity 15 and the respective electrodes becomes at about 750℃ to about 900℃.

In the sensor element 101 of the present embodiment, the heater 72 is embedded in the base part 102, but this form is not limitative. The heater 72 may be disposed to heat the base part 102. That is, the heater 72 may heat the sensor element 101 to develop oxygen ion conductivity with which the main pump cell 21, the first measurement pump cell 50, and the second measurement pump cell 41 are operable. For example, the heater 72 may be embedded in the base part 102 as in the present embodiment. Alternatively, for example, the heater part 70 may be formed as a heater substrate that is separate from the base part 102, and may be disposed at a position adjacent to the base part 102.

The heater insulating layer 74 is formed of an insulator such as alumina on the upper and lower surfaces of the heater 72 and the heater lead 76. The heater insulating layer 74 is formed to ensure electrical insulation between the second substrate layer 2, and the heater 72 and the heater lead 76, and electrical insulation between the third substrate layer 3, and the heater 72 and the heater lead 76.

The pressure relief vent 75 extends through the third substrate layer 3 so that the heater insulating layer 74 and the reference gas introduction space 43 communicate with each other. The pressure relief vent 75 can mitigate an increase in internal pressure due to temperature rise in the heater insulating layer 74. The pressure relief vent 75 may be absent.

Further, an area of a predetermined length in the longitudinal direction in a surface of the sensor element 101 from the front end may be covered by a porous protective layer not shown. The porous protective layer is formed to protect a region where the internal cavities and the electrodes are present in the sensor element 101 from thermal shock caused by water splashing or the like. The porous protective layer may be made of ceramics such alumina, and may have a thickness of approximately 10 μm to 2000 μm. Preferably, the porous protective layer may be formed so as to resist forces up to approximately 50 N.

The above-described sensor element 101 is incorporated into the gas sensor 100 in such a form that the front end part of the sensor element 101 comes into contact with the measurement-object gas, and the rear end part of the sensor element 101 comes into contact with the reference gas.

### Control unit

The gas sensor 100 of this embodiment includes the sensor element 101 described above and the control unit 90 for controlling the sensor element 101. In the gas sensor 100, each of the electrodes 22, 23, 51, 44, and 42 of the sensor element 101 is electrically connected to the control unit 90 through a lead wire not shown. FIG. 2 is a block diagram showing electric connections between the control unit 90 and the respective pump cells 21, 50 and 41, and the respective sensor cells 80, 81, 82, and 83 of the sensor element 101. The control unit 90 includes the above-described variable power supplies 24, 52 and 46, and a control part 91. The control part 91 includes a pump control part 92 and a concentration calculating part 93.

The control part 91 is realized by a general-purpose or dedicated computer, and functions as the pump control part 92 and the concentration calculating part 93 are realized by a CPU, a memory or the like installed in the computer. It is to be noted that when water vapor H₂O and carbon dioxide CO₂ contained in exhaust gas from the engine of a car is target gases to be measured by the gas sensor 100 and the sensor element 101 is attached to an exhaust gas path, some or all of the functions of the control unit 90 (especially, the control part 91) may be realized by an electronic control unit (ECU) installed in the car.

The control part 91 is configured to acquire an electromotive force (voltage V0, V1, V2, Vref) in each of the sensor cells 80, 81, 82, and 83, and a pump current (Ip0, Ip1, Ip2) in each of the pump cells 21, 50 and 41 of the sensor element 101. Further, the control part 91 is configured to output control signals to the variable power supplies 24, 52 and 46.

The pump control part 92 is configured to control the operation of the oxygen pump cell (in this embodiment, the main pump cell 21), the first measurement pump cell 50, and the second measurement pump cell 41 so as to measure a water vapor concentration and a carbon dioxide concentration in a measurement-object gas.

The pump control part 92 is configured to have an oxygen measurement mode and a normal measurement mode.

In the oxygen measurement mode, the pump control part 92 operates the oxygen pump cell (the main pump cell 21) to pump out oxygen from the first internal cavity 20. In the oxygen measurement mode, the pump control part 92 operates the oxygen pump cell (the main pump cell 21) so that oxygen in the first internal cavity 20 is pumped out and none of water vapor and carbon dioxide in the measurement-object gas is decomposed in the first internal cavity 20.

In the oxygen measurement mode, the pump control part 92 may apply a predetermined oxygen pump voltage (pump voltage Vp0) at which neither the water vapor nor the carbon dioxide is decomposed on the intracavity oxygen pump electrode (the inner main pump electrode 22) between the intracavity oxygen pump electrode (the inner main pump electrode 22) and the extracavity oxygen pump electrode (the outer pump electrode 23) of the oxygen pump cell (the main pump cell 21) to pump out oxygen from the first internal cavity 20. The pump control part 92 may perform, for example, the following feedback control based on an electromotive force (a voltage V0) generated in the oxygen-partial-pressure detection sensor cell 80 for main pump control to pump out oxygen from the first internal cavity 20. Specifically, in the present embodiment, control of the oxygen measurement mode is performed in the following manner.

In the oxygen measurement mode, the pump control part 92 performs feedback control of the pump voltage Vp0 of the variable power supply 24 in the main pump cell 21 so that the electromotive force (the voltage V0) generated between the intracavity oxygen pump electrode (the inner main pump electrode 22) and the reference electrode 42 is at a constant value (referred to as a set value V0a_{SET}). The set value V0a_{SET} is set as a control voltage in the oxygen measurement mode with respect to the main pump cell 21. The set value V0a_{SET} may be set as a value such that the oxygen partial pressure of the atmosphere in the first internal cavity 20 is made to be an oxygen partial pressure at which all of, or substantially all of oxygen in the measurement-object gas in the first internal cavity 20 is pumped out and none of, or substantially none of water vapor and carbon dioxide in the measurement-object gas is decomposed in the first internal cavity 20. For example, the set value V0a_{SET} may be set as a value such that the pump current Ip0 is to be a limiting current. The set value V0a_{SET} may vary depending on the intended use of the gas sensor 100, the configuration of the sensor element 101 and the like, and the set value V0a_{SET} may be, for example, about 200 mV or more and about 700 mV or less.

In the normal measurement mode,
the pump control part 92 applies a predetermined oxygen pump voltage (pump voltage Vp0) between the intracavity oxygen pump electrode (the inner main pump electrode 22) and the extracavity oxygen pump electrode (the outer pump electrode 23) of the oxygen pump cell (the main pump cell 21) to pump out oxygen from the first internal cavity 20 and adjust an oxygen partial pressure in the first internal cavity 20 such that substantially all of water vapor and carbon dioxide in the measurement-object gas are decomposed in the first internal cavity 20;
the pump control part 92 applies a predetermined first measurement pump voltage (pump voltage Vp1) between the intracavity first measurement electrode (the first measurement electrode 51) and the extracavity first measurement electrode (the outer pump electrode 23) of the first measurement pump cell 50 to pump oxygen into the second internal cavity 40 and adjust an oxygen partial pressure in the second internal cavity 40 such that hydrogen generated by decomposition of the water vapor is selectively oxidized in the second internal cavity 40; and
the pump control part 92 applies a predetermined second measurement pump voltage (pump voltage Vp2) between the intracavity second measurement electrode (the second measurement electrode 44) and the extracavity second measurement electrode (the outer pump electrode 23) of the second measurement pump cell 41 to pump oxygen into near a surface of the intracavity second measurement electrode (the second measurement electrode 44), namely, into the third internal cavity 61, and adjust an oxygen partial pressure in the third internal cavity 61 such that carbon monoxide generated by decomposition of the carbon dioxide is selectively oxidized near the surface of the intracavity second measurement electrode (the second measurement electrode 44).

The pump control part 92 may adjust the oxygen partial pressure in each of the internal cavities 20, 40, and 61 by adjusting the pump voltage (Vp0, Vp1, Vp2) in each of the pump cells 21, 50, and 41. Further, the pump control part 92 may adjust the oxygen partial pressure in each of the internal cavities 20, 40, and 61 by performing, for example, the following feedback control based on the electromotive force (voltage V0, V1, V2) generated in each of the sensor cells 80, 81, and 82. Specifically, in the present embodiment, control of the normal measurement mode is performed in the following manner.

In the normal measurement mode, the pump control part 92 performs feedback control of the pump voltage Vp0 of the variable power supply 24 in the main pump cell 21 so that the electromotive force (the voltage V0) generated between the intracavity oxygen pump electrode (the inner main pump electrode 22) and the reference electrode 42 is at a constant value (referred to as a set value V0_{SET}). The set value V0_{SET} is set as a control voltage in the normal measurement mode with respect to the main pump cell 21. The set value V0_{SET} may be set as a value such that the oxygen partial pressure of the atmosphere in the first internal cavity 20 is made to be an oxygen partial pressure at which all of, or substantially all of the water vapor and the carbon dioxide in the measurement-object gas is decomposed in the first internal cavity 20. The set value V0_{SET} may vary depending on the intended use of the gas sensor 100, the configuration of the sensor element 101 and the like, and the set value V0_{SET} may be, for example, about 1200 mV or more and about 1500 mV or less, or, about 1300 mV or more and about 1500 mV or less.

In the normal measurement mode, the pump control part 92 performs feedback control of the pump voltage Vp1 of the variable power supply 52 in the first measurement pump cell 50 so that the electromotive force (the voltage V1) generated between the intracavity first measurement electrode (the first measurement electrode 51) and the reference electrode 42 is at a constant value (referred to as a set value V1_{SET}). The set value V1_{SET} may be set as a value such that the oxygen partial pressure of the atmosphere in the second internal cavity 40 is made to be an oxygen partial pressure at which the hydrogen generated by the decomposition of the water vapor is selectively burned in the second internal cavity 40. That is, the set value V1_{SET} may be set as a value such that the oxygen partial pressure of the atmosphere in the second internal cavity 40 is made to be an oxygen partial pressure at which all of, or substantially all of the hydrogen generated by the decomposition of the water vapor is burned in the vicinity of the surface of the intracavity first measurement electrode (the first measurement electrode 51) in the second internal cavity 40, and the carbon monoxide generated by the decomposition of the carbon dioxide is not burned, or not substantially burned in the vicinity of the surface of the intracavity first measurement electrode (the first measurement electrode 51) in the second internal cavity 40. The phrase “not substantially burned” includes that burning of the carbon monoxide at the intracavity first measurement electrode (the first measurement electrode 51) is so low as not to affect measurement of a carbon dioxide concentration in the gas sensor 100, or that a combustion ratio of the carbon monoxide is constant. The set value V1_{SET} may vary depending on the intended use of the gas sensor 100, the configuration of the sensor element 101 and the like, and the set value V1_{SET} may be, for example, about 200 mV or more and about 700 mV or less. The set value V1_{SET} may be, for example, about 350 mV.

In the normal measurement mode, the pump control part 92 performs feedback control of the pump voltage Vp2 of the variable power supply 46 in the second measurement pump cell 41 so that the electromotive force (the voltage V2) generated between the intracavity second measurement electrode (the second measurement electrode 44) and the reference electrode 42 is at a constant value (referred to as a set value V2_{SET}). The set value V2_{SET} may be set as a value such that the oxygen partial pressure of the atmosphere in the vicinity of the surface of the intracavity second measurement electrode (the second measurement electrode 44), namely, in the third internal cavity 61 is made to be an oxygen partial pressure at which the carbon monoxide generated by the decomposition of the carbon dioxide is selectively burned in the vicinity of the surface of the intracavity second measurement electrode (the second measurement electrode 44). That is, the set value V2_{SET} may be set as a value such that the oxygen partial pressure in the vicinity of the surface of the intracavity second measurement electrode (the second measurement electrode 44) is made to be an oxygen partial pressure at which all of, or substantially all of the carbon monoxide generated by the decomposition of the carbon dioxide is burned in the vicinity of the surface of the intracavity second measurement electrode (the second measurement electrode 44). The set value V2_{SET} is generally a value smaller than the set value V1_{SET}. The set value V2_{SET} may vary depending on the intended use of the gas sensor 100, the configuration of the sensor element 101 and the like, and the set value V2_{SET} may be, for example, about 150 mV or more and about 400 mV or less. The set value V2_{SET} may be, for example, about 200 mV.

The concentration calculating part 93 is configured to calculate a water vapor concentration and a carbon dioxide concentration in a measurement-object gas.

The concentration calculating part 93 calculates an oxygen concentration in the oxygen measurement mode;
the concentration calculating part 93 calculates a water vapor concentration in the measurement-object gas based on a current (a pump current Ip1) flowing through the first measurement pump cell 50 in the normal measurement mode and the oxygen concentration in the oxygen measurement mode; and
the concentration calculating part 93 calculates a carbon dioxide concentration in the measurement-object gas based on a current (a pump current Ip2) flowing through the second measurement pump cell 41 in the normal measurement mode and the oxygen concentration in the oxygen measurement mode.

The concentration calculating part 93 acquires the pump current Ip0 flowing through the oxygen pump cell (the main pump cell 21) in the oxygen measurement mode, and calculates O₂ concentration in the measurement-object gas in the oxygen measurement mode on the basis of a previously-stored conversion parameter (current-O₂ concentration conversion parameter) between the pump current Ip0 and the oxygen concentration (O₂ concentration) in the measurement-object gas. The current-O₂ concentration conversion parameter is previously stored, as data showing a relationship between the pump current Ip0 and the O₂ concentration in the measurement-object gas, in the memory of the control part 91 which functions as the concentration calculating part 93. The current-O₂ concentration conversion parameter may appropriately be determined by those skilled in the art by, for example, previously performing an experiment on the gas sensor 100. The current-O₂ concentration conversion parameter may be, for example, the coefficient of an approximate expression (e.g., linear function) obtained by experiment or a map showing the relationship between the pump current Ip0 and the O₂ concentration in a measurement-object gas. The current-O₂ concentration conversion parameter may be specific to each individual gas sensor 100 or may be common to a plurality of gas sensors.

In the present embodiment,
the concentration calculating part 93 corrects the current (pump current Ip1) flowing through the first measurement pump cell 50 in the normal measurement mode based on the calculated O₂ concentration in the measurement-object gas in the oxygen measurement mode to calculate a corrected current (a corrected pump current Ip1), and calculates the water vapor concentration in the measurement-object gas based on the corrected current (the corrected pump current Ip1); and
the concentration calculating part corrects the current (pump current Ip2) flowing through the second measurement pump cell 41 in the normal measurement mode based on the calculated O₂ concentration in the measurement-object gas in the oxygen measurement mode to calculate a corrected current (a corrected pump current Ip2), and calculates the carbon dioxide concentration in the measurement-object gas based on the corrected current (the corrected pump current Ip2). In this embodiment, the correction is to calculate a correction value with respect to the pump current Ip1 and a correction value with respect to the pump current Ip2 based on the O₂ concentration in the measurement-object gas, and to calculate the respective corrected currents using the calculated correction values.

The concentration calculating part 93 calculates a correction value with respect to the pump current Ip1 on the basis of the calculated O₂ concentration in the measurement-object gas in the oxygen measurement mode, and a previously-stored correspondence (O₂ concentration-H₂O correction value correspondence) between the O₂ concentration in the measurement-object gas and the correction value with respect to the pump current Ip1. The O₂ concentration-H₂O correction value correspondence is previously stored, as data showing a relationship between the O₂ concentration in the measurement-object gas and the correction value with respect to the pump current Ip1 as will be described later, in the memory of the control part 91 which functions as the concentration calculating part 93. The O₂ concentration-H₂O correction value correspondence may appropriately be determined by those skilled in the art by, for example, previously performing an experiment on the gas sensor 100. The O₂ concentration-H₂O correction value correspondence may be, for example, a deviation amount from an ideal value of the pump current Ip1 obtained by experiment, the coefficient of a function when the correction value with respect to the pump current Ip1 is represented as a function of O₂ concentration, or a map showing the relationship between the O₂ concentration and the correction value with respect to the pump current Ip1. The O₂ concentration-H₂O correction value correspondence may be specific to each individual gas sensor 100 or may be common to a plurality of gas sensors.

The concentration calculating part 93 acquires the pump current Ip1 in the first measurement pump cell 50, and corrects the pump current Ip1 by using the above calculated correction value with respect to the pump current Ip1 to calculate a corrected pump current Ip1. The corrected pump current Ip1 is to be, regardless of the oxygen concentration, a value corrected to a current value that should flow at a standard oxygen concentration (for example, oxygen concentration of 0%). The concentration calculating part 93 calculates H₂O concentration in the measurement-object gas on the basis of the calculated corrected pump current Ip1, and a previously-stored conversion parameter (current-H₂O concentration conversion parameter) between the pump current Ip1 and the water vapor concentration (H₂O concentration) in the measurement-object gas. The concentration calculating part 93 outputs the calculated H₂O concentration as a measurement value of the gas sensor 100. The current-H₂O concentration conversion parameter is previously stored, as data showing a relationship between the pump current Ip1 at the standard oxygen concentration (for example, oxygen concentration of 0%) and the H₂O concentration in the measurement-object gas, in the memory of the control part 91 which functions as the concentration calculating part 93. The current-H₂O concentration conversion parameter may appropriately be determined by those skilled in the art by, for example, previously performing an experiment on the gas sensor 100. The current-H₂O concentration conversion parameter may be, for example, the coefficient of an approximate expression (e.g., linear function) obtained by experiment or a map showing the relationship between the pump current Ip1 and the H₂O concentration in a measurement-object gas. The current-H₂O concentration conversion parameter may be specific to each individual gas sensor 100 or may be common to a plurality of gas sensors.

The concentration calculating part 93 calculates a correction value with respect to the pump current Ip2 on the basis of the calculated O₂ concentration in the measurement-object gas in the oxygen measurement mode, and a previously-stored correspondence (O₂ concentration-CO₂correction value correspondence) between the O₂ concentration in the measurement-object gas and the correction value with respect to the pump current Ip2. The O₂ concentration-CO₂ correction value correspondence is previously stored, as data showing a relationship between the O₂ concentration in the measurement-object gas and the correction value with respect to the pump current Ip2 as will be described later, in the memory of the control part 91 which functions as the concentration calculating part 93. The O₂ concentration-CO₂ correction value correspondence may appropriately be determined by those skilled in the art by, for example, previously performing an experiment on the gas sensor 100. The O₂ concentration-CO₂ correction value correspondence may be, for example, a deviation amount from an ideal value of the pump current Ip2 obtained by experiment, the coefficient of a function when the correction value with respect to the pump current Ip2 is represented as a function of O₂ concentration, or a map showing the relationship between the O₂ concentration and the correction value with respect to the pump current Ip2. The O₂ concentration-CO₂ correction value correspondence may be specific to each individual gas sensor 100 or may be common to a plurality of gas sensors.

The concentration calculating part 93 acquires the pump current Ip2 in the second measurement pump cell 41, and corrects the pump current Ip2 by using the above calculated correction value with respect to the pump current Ip2 to calculate a corrected pump current Ip2. The corrected pump current Ip2 is to be, regardless of the oxygen concentration, a value corrected to a current value that should flow at a standard oxygen concentration (for example, oxygen concentration of 0%). The concentration calculating part 93 calculates CO₂ concentration in the measurement-object gas on the basis of the calculated corrected pump current Ip2, and a previously-stored conversion parameter (current-CO₂ concentration conversion parameter) between the pump current Ip2 and the carbon dioxide concentration (CO₂ concentration) in the measurement-object gas. The concentration calculating part 93 outputs the calculated CO₂ concentration as a measurement value of the gas sensor 100. The current-CO₂ concentration conversion parameter is previously stored, as data showing a relationship between the pump current Ip2 at the standard oxygen concentration (for example, oxygen concentration of 0%) and the CO₂ concentration in the measurement-object gas, in the memory of the control part 91 which functions as the concentration calculating part 93. The current-CO₂ concentration conversion parameter may appropriately be determined by those skilled in the art by, for example, previously performing an experiment on the gas sensor 100. The current-CO₂ concentration conversion parameter may be, for example, the coefficient of an approximate expression (e.g., linear function) obtained by experiment or a map showing the relationship between the pump current Ip2 and the CO₂ concentration in a measurement-object gas. The current-CO₂ concentration conversion parameter may be specific to each individual gas sensor 100 or may be common to a plurality of gas sensors.

### Water vapor detecting current and carbon dioxide detecting current

The water vapor detecting current (namely, the pump current Ip1) and the carbon dioxide detecting current (namely, the pump current Ip2) will be described in detail.

As described above, in the normal measurement mode, the pump control part 92 decomposes water vapor and carbon dioxide in the measurement-object gas in first internal cavity 20, selectively oxidizes hydrogen generated by decomposition of the water vapor in the second internal cavity 40, and selectively oxidizes carbon monoxide generated by decomposition of the carbon dioxide in the third internal cavity 61.

It is found that the water vapor detecting current (the pump current Ip1) and the carbon dioxide detecting current (the pump current Ip2) obtained as a result of such pump control in the normal measurement mode vary depending on the oxygen concentration in the measurement-object gas, that is, receive interference from the oxygen concentration. This property of being affected from the oxygen concentration is also referred to as oxygen interference (O₂ interference).

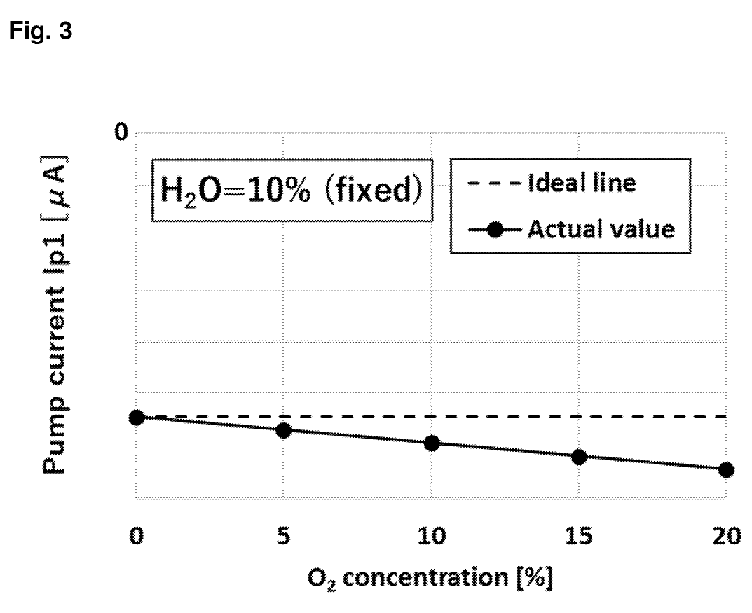

FIG. 3 is a graph showing one example of a relationship between oxygen concentration (O₂ concentration) in the measurement-object gas and the pump current Ip1. The horizontal axis of the graph represents the O₂ concentration [%] and the vertical axis of the graph represents the pump current Ip1 [μA]. The pump current Ip1 is a current for pumping oxygen into the second internal cavity 40, and is represented as a negative current. As the measurement-object gas, model gases having oxygen concentrations of 0, 5, 10, 15, and 20% are used. The gas composition other than oxygen is water vapor concentration of 10%, carbon dioxide concentration of 0% and nitrogen (reminder) in each of the model gases. “%” indicates % by volume in all cases. The same applies hereinafter.

Ideally, the pump current Ip1 is preferably constant regardless of oxygen concentration, as an ideal line illustrated by a broken line in FIG. 3, when water vapor concentration is constant. However, actually, the pump current Ip1 varies depending on the oxygen concentration, as illustrated by a solid line in FIG. 3. Specifically, the pump current Ip1 becomes negatively larger (namely, larger as an absolute value) as the oxygen concentration in the measurement-object gas becomes higher, even when the water vapor concentration is constant (10% in FIG. 3). That is, oxygen pumped into the second internal cavity 40 by the first measurement pump cell 50 increases as the oxygen concentration in the measurement-object gas becomes higher, even when the water vapor concentration is constant (10% in FIG. 3).

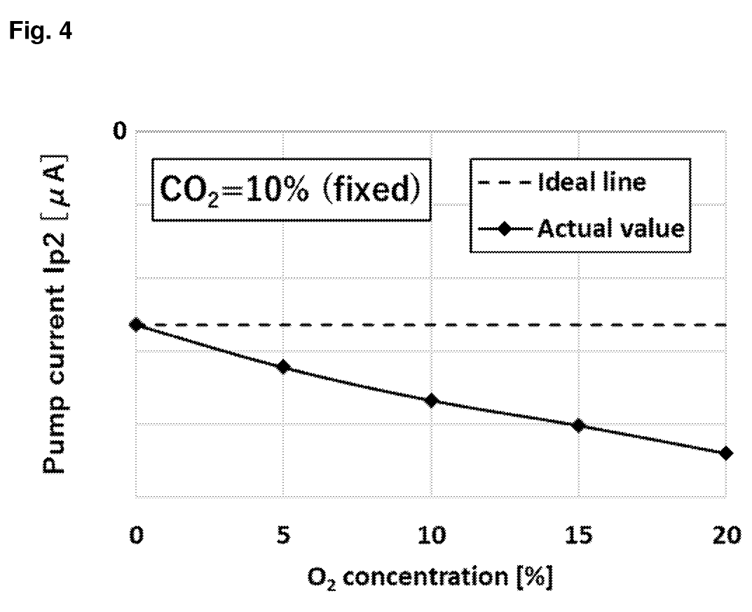

FIG. 4 is a graph showing one example of a relationship between oxygen concentration (O₂ concentration) in the measurement-object gas and the pump current Ip2. The horizontal axis of the graph represents the O₂ concentration [%] and the vertical axis of the graph represents the pump current Ip2 [μA]. The pump current Ip2 is a current for pumping oxygen into the third internal cavity 61, and is represented as a negative current. As the measurement-object gas, model gases having oxygen concentrations of 0, 5, 10, 15, and 20% are used. The gas composition other than oxygen is water vapor concentration of 0%, carbon dioxide concentration of 10% and nitrogen (reminder) in each of the model gases.

Ideally, the pump current Ip2 is preferably constant regardless of oxygen concentration, as an ideal line illustrated by a broken line in FIG. 4, when carbon dioxide concentration is constant. However, actually, the pump current Ip2 varies depending on the oxygen concentration, as illustrated by a solid line in FIG. 4. Specifically, the pump current Ip2 becomes negatively larger (namely, larger as an absolute value) as the oxygen concentration in the measurement-object gas becomes higher, even when the carbon dioxide concentration is constant (10% in FIG. 4). That is, oxygen pumped into the third internal cavity 61 by the second measurement pump cell 41 increases as the oxygen concentration in the measurement-object gas becomes higher, even when the carbon dioxide concentration is constant (10% in FIG. 4).

The present inventors consider a mechanism that the pump current Ip1 and the pump current Ip2 vary depending on oxygen concentration as follows. Consideration will be made in the case that a measurement-object gas contains three components, namely, oxygen O₂, water vapor H₂O, and carbon dioxide CO₂. The measurement-object gas is introduced into the first internal cavity 20, and in the first internal cavity 20, oxygen in the measurement-object gas is pumped out by the main pump cell 21. This makes a total gas amount in the first internal cavity 20 decrease, and thus the measurement-object gas in an amount equivalent to the amount of the pumped out oxygen is newly introduced into the first internal cavity 20. The three components, namely, the oxygen O₂, the water vapor H₂O, and the carbon dioxide CO₂ are also present in the newly introduced measurement-object gas. Oxygen in the newly introduced measurement-object gas is also pumped out by the main pump cell 21, and the measurement-object gas in an amount equivalent to the amount of the pumped out oxygen is further introduced into the first internal cavity 20. Oxygen in the further introduced measurement-object gas is also pumped out by the main pump cell 21. By repetition thereof, the water vapor concentration and the carbon dioxide concentration in the gas present in the first internal cavity 20 is considered to increase compared with the water vapor concentration and the carbon dioxide concentration in the measurement-object gas, respectively, when the measurement-object gas contains oxygen. As a result, it is considered that since an amount of hydrogen generated by decomposition of water vapor in the first internal cavity 20 increases to increase oxygen to be pumped in for burning (oxidizing) the hydrogen in the second internal cavity 40, an absolute value of the pump current Ip1 increases. It is also considered that since an amount of carbon monoxide generated by decomposition of carbon dioxide in the first internal cavity 20 increases to increase oxygen to be pumped in for burning (oxidizing) the carbon monoxide in the third internal cavity 61, an absolute value of the pump current Ip2 increases.

It is considered that the water vapor concentration in the measurement-object gas can be measured more accurately by eliminating the above-described effect due to the oxygen concentration from the pump current Ip1. It is also considered that the carbon dioxide concentration in the measurement-object gas can be measured more accurately by eliminating the above-described effect due to the oxygen concentration from the pump current Ip2.

As a result of intensive studies, the present inventors have found that a water vapor concentration and a carbon dioxide concentration in a measurement-object gas can be measured more accurately by performing an oxygen measurement mode for measuring an oxygen concentration in the measurement-object gas, and a normal measurement mode for measuring a pump current Ip1 flowing depending on the water vapor concentration and a pump current flowing depending on the carbon dioxide concentration in the measurement-object gas. The present inventors have found that the water vapor concentration can be measured more accurately by calculating the water vapor concentration based on the pump current Ip1 in the normal measurement mode and the oxygen concentration measured in the oxygen measurement mode. The present inventors have also found that the carbon dioxide concentration can be measured more accurately by calculating the carbon dioxide concentration based on the pump current Ip2 in the normal measurement mode and the oxygen concentration measured in the oxygen measurement mode.

For example, as described above, the concentration calculating part 93 may correct the current (pump current Ip1) flowing through the first measurement pump cell 50 based on the O₂concentration in the measurement-object gas to calculate a corrected current (corrected pump current Ip1), and may calculate the water vapor concentration in the measurement-object gas based on the corrected current (corrected pump current Ip1); and
the concentration calculating part may correct the current (pump current Ip2) flowing through the second measurement pump cell 41 based on the O₂ concentration in the measurement-object gas to calculate a corrected current (corrected pump current Ip2), and may calculate the carbon dioxide concentration in the measurement-object gas based on the corrected current (corrected pump current Ip2).

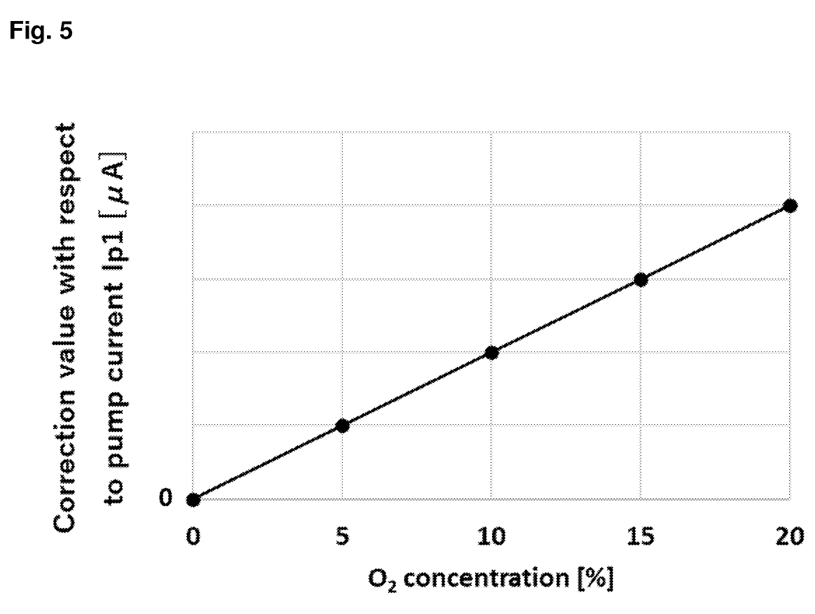

For example, a deviation amount from the ideal line of the pump current Ip1 in FIG. 3 may be used as a correction value with respect to the pump current Ip1. A corrected pump current Ip1 may be calculated by adding the correction value to the pump current Ip1 that is a negative current. The corrected pump current Ip1 is to be a value on the ideal line in FIG. 3. FIG. 5 is a graph showing one example of a relationship between oxygen concentration (O₂ concentration) in the measurement-object gas and the correction value with respect to the pump current Ip1. The horizontal axis of the graph represents the O₂ concentration [%] and the vertical axis of the graph represents the correction value with respect to the pump current Ip1 [μA]. In FIG. 5, the correction value with respect to the pump current Ip1 (also referred to as a H₂O correction value) is the deviation amount from the ideal line of the pump current Ip1 in FIG. 3, that is, a difference by subtracting an actual pump current Ip1 from a value on the ideal line in each of the oxygen concentration. As shown in FIG. 5, the correction value with respect to the pump current Ip1 has a correspondence with the oxygen concentration. In other words, the correction value with respect to the pump current Ip1 is expressed as a function of the oxygen concentration. The concentration calculating part 93 may previously store such a correspondence (O₂ concentration-H₂O correction value correspondence) between the oxygen concentration and the correction value with respect to the pump current Ip1.

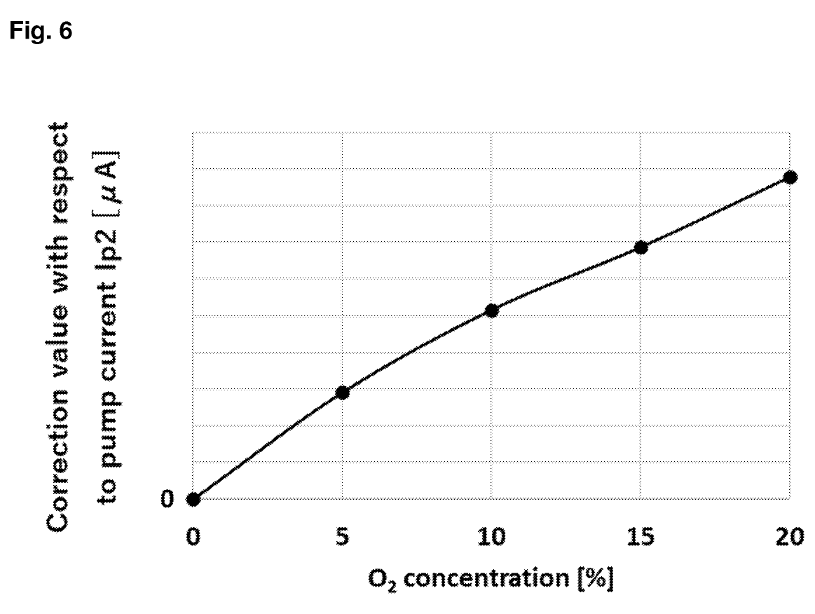

For example, a deviation amount from the ideal line of the pump current Ip2 in FIG. 4 may be used as a correction value with respect to the pump current Ip2. A corrected pump current Ip2 may be calculated by adding the correction value to the pump current Ip2 that is a negative current. The corrected pump current Ip2 is to be a value on the ideal line in FIG. 4. FIG. 6 is a graph showing one example of a relationship between oxygen concentration (O₂ concentration) in the measurement-object gas and the correction value with respect to the pump current Ip2. The horizontal axis of the graph represents the O₂ concentration [%] and the vertical axis of the graph represents the correction value with respect to the pump current Ip2 [μA]. In FIG. 6, the correction value with respect to the pump current Ip2 (also referred to as a CO₂ correction value) is the deviation amount from the ideal line of the pump current Ip2 in FIG. 4, that is, a difference by subtracting an actual pump current Ip2 from a value on the ideal line in each of the oxygen concentration. As shown in FIG. 6, the correction value with respect to the pump current Ip2 has a correspondence with the oxygen concentration. In other words, the correction value with respect to the pump current Ip2 is expressed as a function of the oxygen concentration. The concentration calculating part 93 may previously store such a correspondence (O₂ concentration-CO₂ correction value correspondence) between the oxygen concentration and the correction value with respect to the pump current Ip2.

### Measurement of water vapor concentration and carbon dioxide concentration

Next, a method for measuring each concentration of water vapor H₂O and carbon dioxide CO₂ in the measurement-object gas by using the gas sensor 100 having such a configuration as described above will be described.

A control method of a gas sensor for measuring a concentration of each of water vapor H₂O and carbon dioxide CO₂ in a measurement-object gas includes:
an oxygen concentration measuring step of calculating an oxygen concentration in an oxygen measurement mode;
a water vapor concentration measuring step of calculating a water vapor concentration in the measurement-object gas based on a current (pump current Ip1) flowing through the first measurement pump cell 50 in the normal measurement mode and the oxygen concentration in the oxygen measurement mode; and
a carbon dioxide concentration measuring step of calculating a carbon dioxide concentration in the measurement-object gas based on a current (pump current Ip2) flowing through the second measurement pump cell 41 in the normal measurement mode and the oxygen concentration in the oxygen measurement mode.

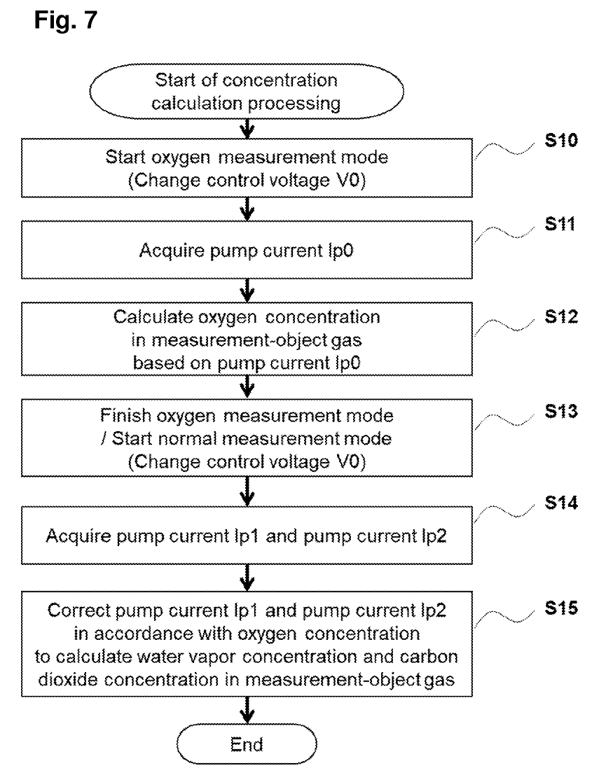

FIG. 7 is a flow chart of concentration calculating processing of each of water vapor H₂O and carbon dioxide CO₂ in a measurement-object gas.

When the concentration calculating processing is started, the pump control part 92 starts the oxygen measurement mode (step S10). The pump control part 92 sets the control voltage in the main pump cell 21 to the set value V0a_{SET}, and performs the oxygen measurement mode as described above.

That is, the pump control part 92 uses the voltage V0 generated between the inner main pump electrode 22 and the reference electrode 42 as the control voltage, and performs feedback control of the pump voltage Vp0 of the variable power supply 24 in the main pump cell 21 so that the voltage V0 is at the set value V0a_{SET}.

In the oxygen measurement mode, by operating the main pump cell 21 as described above, the pump control part 92 pumps out all of, or substantially all of oxygen in the measurement-object gas introduced into the first internal cavity 20. In the oxygen measurement mode, none of, or substantially none of water vapor and carbon dioxide in the measurement-object gas is decomposed. Therefore, the pump current Ip0 flowing through the main pump cell 21 is to be a value corresponding to the oxygen concentration in the measurement-object gas.

It is to be noted that the pump control part 92 may control, or may not control operation of the first measurement pump cell 50 and the second measurement pump cell 41 in the oxygen measurement mode. For example, with respect to the first measurement pump cell 50 and the second measurement pump cell 41, the pump control part 92 may adopt the same control as in the case of the normal measurement mode. In this case, since decomposition of water vapor and carbon dioxide does not occur in the first internal cavity 20 in the oxygen measurement mode, the pump current Ip1 flowing through the first measurement pump cell 50 may be at a value equivalent to a value when water vapor is not present in the measurement-object gas in the normal measurement mode. Further, the pump current Ip2 flowing through the second measurement pump cell 41 may be at a value equivalent to a value when carbon dioxide is not present in the measurement-object gas in the normal measurement mode.

Next, the concentration calculating part 93 acquires the pump current Ip0 in the oxygen measurement mode (step S11). And, the concentration calculating part 93 calculates oxygen concentration in the measurement-object gas on the basis of the acquired pump current Ip0 (step S12). Specifically, the concentration calculating part 93 calculates O₂ concentration in the measurement-object gas in the oxygen measurement mode on the basis of the acquired pump current Ip0, and a previously-stored conversion parameter (current-O₂ concentration conversion parameter) between the pump current Ip0 and the oxygen concentration (O₂ concentration) in the measurement-object gas.

The pump control part 92 finishes the oxygen measurement mode and starts the normal measurement mode (step S13). The pump control part 92 changes the control voltage in the main pump cell 21 form the set value V0a_{SET} to the set value V0_{SET}, and performs the normal measurement mode as described above.

That is, the pump control part 92 uses the voltage V0 generated between the inner main pump electrode 22 and the reference electrode 42 as the control voltage, and performs feedback control of the pump voltage Vp0 of the variable power supply 24 in the main pump cell 21 so that the voltage V0 is at the set value V0_{SET};
the pump control part 92 performs feedback control of the pump voltage Vp1 of the variable power supply 52 in the first measurement pump cell 50 so that the voltage V1 generated between the first measurement electrode 51 and the reference electrode 42 is at the set value V1SET; and
the pump control part 92 performs feedback control of the pump voltage Vp2 of the variable power supply 46 in the second measurement pump cell 41 so that the voltage V2 generated between the second measurement electrode 44 and the reference electrode 42 is at the set value V2SET.

In the normal measurement mode, by operating the main pump cell 21 as described above, the pump control part 92 pumps out oxygen from the first internal cavity 20 so that the oxygen partial pressure of the measurement-object gas introduced into the first internal cavity 20 is made to be a low oxygen partial pressure (for example, about 10⁻¹⁰ to 10⁻³⁰ atm) at which all of, or substantially all of water vapor and carbon dioxide contained in the measurement-object gas are decomposed. “All of, or substantially all of water vapor and carbon dioxide contained in the measurement-object gas are decomposed” means that the water vapor and the carbon dioxide introduced into the first internal cavity 20 are not substantially introduced into the second internal cavity 40.

When oxygen is pumped out from the first internal cavity 20 in such a manner, the decomposition reaction of water vapor (2H₂O → 2H₂ + O₂) and the decomposition reaction of carbon dioxide (2CO₂ → 2CO + O₂) are promoted in the first internal cavity 20 (especially, in the vicinity of the surface of the inner main pump electrode 22). Hydrogen and oxygen are generated by the decomposition of the water vapor, and carbon monoxide and oxygen are generated by the decomposition of the carbon dioxide. The generated oxygen is pumped out together with oxygen gas originally present in the measurement-object gas by the main pump cell 21. On the other hand, the hydrogen and the carbon monoxide are introduced into the second internal cavity 40.

The set value V0_{SET} of the voltage V0 may be preferably set such that an oxygen partial pressure (a target oxygen partial pressure) after adjusted in the first internal cavity 20 becomes smaller as the oxygen partial pressure in the measurement-object gas that has reached the first internal cavity 20 becomes larger. This may allow oxygen to be pumped out more reliably.

The measurement-object gas, which contains the hydrogen generated by the decomposition of the water vapor and the carbon monoxide generated by the decomposition of the carbon dioxide, passes through the second diffusion-rate limiting path 30 so that a predetermined diffusion resistance is imparted to the measurement-object gas, and reaches the second internal cavity 40.

By operating the first measurement pump cell 50 as described above, the pump control part 92 pumps oxygen into the second internal cavity 40. Consequently, hydrogen in the measurement-object gas containing hydrogen and carbon monoxide, which has reached the vicinity of the surface of the first measurement electrode 51 in the second internal cavity 40, selectively react with oxygen existing at this position for burning (2H₂ + O₂ → 2H₂O). Oxygen may be pumped in by the first measurement pump cell 50 so that substantially all of the hydrogen having reached the vicinity of the surface of the first measurement electrode 51 is burned. Thus, in the measurement-object gas, the hydrogen generated by the decomposition of the water vapor is not present, and the carbon monoxide generated by the decomposition of the carbon dioxide is contained. For example, oxygen may be pumped in by the first measurement pump cell 50 so that the oxygen partial pressure in the second internal cavity 40, especially, in the vicinity of the surface of the first measurement electrode 51, is made to be at 10⁻⁵ atm to 10⁻¹⁵ atm.

The pump current (water vapor detecting current) Ip1 flowing through the first measurement pump cell 50 at this time is substantially proportional to an amount (or, a concentration) of the hydrogen burning in the vicinity of the surface of the first measurement electrode 51. An amount of hydrogen having reached the vicinity of the surface of the first measurement electrode 51 is correlated with an amount of water vapor contained in the measurement-object gas introduced through the gas inlet 10.

The measurement-object gas, in which the hydrogen generated by the decomposition of the water vapor has been burned and the carbon monoxide generated by the decomposition of the carbon dioxide is contained, passes through the third diffusion-rate limiting path 60 so that a predetermined diffusion resistance is imparted to the measurement-object gas, and reaches the third internal cavity 61.

By operating the second measurement pump cell 41 as described above, the pump control part 92 pumps oxygen into the third internal cavity 61. Consequently, carbon monoxide in the measurement-object gas containing the carbon monoxide, which has reached the vicinity of the surface of the second measurement electrode 44 in the third internal cavity 61, selectively react with oxygen existing at this position for burning (2CO + O₂ → 2CO₂). Oxygen may be pumped in by the second measurement pump cell 41 so that substantially all of the carbon monoxide having reached the vicinity of the surface of the second measurement electrode 44 is burned. For example, oxygen may be pumped in by the second measurement pump cell 41 so that the oxygen partial pressure in the third internal cavity 61, especially, in the vicinity of the surface of the second measurement electrode 44, is made to be at 10⁻⁵ atm to 10⁻¹⁵atm.

The pump current (carbon dioxide detecting current) Ip2 flowing through the second measurement pump cell 41 at this time is substantially proportional to an amount (or, a concentration) of the carbon monoxide burning in the vicinity of the surface of the second measurement electrode 44. An amount of carbon monoxide having reached the vicinity of the surface of the second measurement electrode 44 is correlated with an amount of carbon dioxide contained in the measurement-object gas introduced through the gas inlet 10.

If the measurement-object gas introduced through the gas inlet 10 contains no water vapor, decomposition of water vapor does not occur in the first internal cavity 20, and thus, hydrogen is not introduced into the second internal cavity 40. However, to keep the voltage V1 at the set value V1_{SET}, a slight amount of the water vapor detecting current Ip1 (so-called an offset current) generally flows. Also, if the measurement-object gas introduced through the gas inlet 10 contains no carbon dioxide, decomposition of carbon dioxide does not occur in the first internal cavity 20, and thus, carbon monoxide is not introduced into the third internal cavity 61. However, to keep the voltage V2 at the set value V2_{SET}, a slight amount of the carbon dioxide detecting current Ip2 (so-called an offset current) generally flows.

The above-described manner of controlling an oxygen partial pressure facilitates selective burning of hydrogen near the surface of the first measurement electrode 51 and selective burning of carbon monoxide on the surface of the second measurement electrode 44. This is because due to a difference in gas diffusion rate between hydrogen and carbon monoxide, hydrogen has a higher gas diffusion rate and is more likely to come into contact with oxygen for burning than carbon monoxide, and hydrogen is more likely to combine with oxygen, that is, is more likely to burn, than carbon monoxide.

Next, the concentration calculating part 93 acquires the pump current Ip1 and the pump current Ip2 in the normal measurement mode (step S14). And, the concentration calculating part 93 corrects the acquired pump current Ip1 and pump current Ip2 in accordance with the oxygen concentration calculated in step S12, and calculates a water vapor concentration and a carbon dioxide concentration in the measurement-object gas (step S15). Specifically, the correction and the concentration calculation is performed in the following manner.

The concentration calculating part 93 calculates a correction value with respect to the pump current Ip1 (H₂O correction value) on the basis of the oxygen concentration calculated in step S12, and a previously-stored correspondence (O₂ concentration-H₂O correction value correspondence) between the oxygen concentration and the correction value with respect to the pump current Ip1. The concentration calculating part 93 corrects the acquired pump current Ip1 by using the calculated H₂O correction value (for example, adds the correction value to the pump current Ip1) to calculate a corrected pump current Ip1. The concentration calculating part 93 calculates a water vapor concentration in the measurement-object gas on the basis of the calculated corrected pump current Ip1, and a previously-stored conversion parameter (current-H₂O concentration conversion parameter) between the pump current Ip1 and the water vapor concentration (H₂O concentration) in the measurement-object gas. The concentration calculating part 93 outputs the calculated water vapor concentration as a measurement value of the gas sensor 100.

The concentration calculating part 93 calculates a correction value with respect to the pump current Ip2 (CO₂correction value) on the basis of the oxygen concentration calculated in step S12, and a previously-stored correspondence (O₂concentration-CO₂ correction value correspondence) between the oxygen concentration and the correction value with respect to the pump current Ip2. The concentration calculating part 93 corrects the acquired pump current Ip2 by using the calculated CO₂ correction value (for example, adds the correction value to the pump current Ip2) to calculate a corrected pump current Ip2. The concentration calculating part 93 calculates a carbon dioxide concentration in the measurement-object gas on the basis of the calculated corrected pump current Ip2, and a previously-stored conversion parameter (current-CO₂ concentration conversion parameter) between the pump current Ip2 and the carbon dioxide concentration (CO₂ concentration) in the measurement-object gas. The concentration calculating part 93 outputs the calculated carbon dioxide concentration as a measurement value of the gas sensor 100.

The gas sensor 100 may repeatedly perform the above-described concentration calculating processing. That is, the pump control part 92 may repeatedly perform the oxygen measurement mode and the normal measurement mode.

In the case of repeating the oxygen measurement mode and the normal measurement mode,
the concentration calculating part 93 may calculate a water vapor concentration in the measurement-object gas based on a pump current Ip1 flowing through the first measurement pump cell 50 in the normal measurement mode and the oxygen concentration in the oxygen measurement mode just before said normal measurement mode; and
the concentration calculating part 93 may calculate a carbon dioxide concentration in the measurement-object gas based on a pump current Ip2 flowing through the second measurement pump cell 41 in the normal measurement mode and the oxygen concentration in the oxygen measurement mode just before said normal measurement mode. As the point of time when the pump current Ip1 and the pump current Ip2 are acquired and the point of time when the oxygen concentration to be used for the correction is calculated are closer, the correction can be done more accurately, and the water vapor concentration and the carbon dioxide concentration can be measured more accurately.

A duration time of the oxygen measurement mode may vary depending on the use environment, required accuracy, and the like of the gas sensor 100, and may be about 0.1 second or more and about 3 seconds or less. When the duration time is within such a range, the pump current Ip0 flowing depending on the oxygen concentration can be measured more accurately, and the pump current Ip1 and the pump current Ip2 in the normal measurement mode can be corrected further precisely. For example, the duration time of the oxygen measurement mode may be about 0.3 second or more, about 0.5 second or more, or about 1 second or more, and may be about 2.5 seconds or less, about 2 seconds or less, or about 1.5 seconds or less.

A duration time of the normal measurement mode may vary depending on the use environment, required accuracy, and the like of the gas sensor 100, and may be about 1 second or more and about 20 seconds or less. When the duration time is within such a range, the pump current Ip1 and the pump current Ip2 can be measured more accurately, and the pump current Ip1 and the pump current Ip2 can be corrected further precisely. For example, the duration time of the normal measurement mode may be about 2 seconds or more, about 3 seconds or more, or about 5 seconds or more, and may be about 18 seconds or less, about 15 seconds or less, or about 10 seconds or less.

In the gas sensor 100, the pump control part 92 performs both the oxygen measurement mode and the normal measurement mode. In the gas sensor 100, the main pump cell 21 functions as a pump cell for pumping out only oxygen in a measurement-object gas in the oxygen measurement mode, and functions as a pump cell for decomposing water vapor and carbon dioxide in the normal measurement mode. Since the main pump cell 21 serves two functions in this way, it is not necessary to form a separate electrode and a separate cell for measuring the oxygen concentration. Therefore, without complicating the structure of the sensor element 101, the water vapor concentration and the carbon dioxide concentration in the measurement-object gas can be measured more accurately, regardless of the oxygen concentration in the measurement-object gas.

The oxygen measurement mode is the control of pumping out oxygen from the first internal cavity 20 while water vapor and carbon dioxide is not decomposed in the first internal cavity 20 to make it possible to measure the oxygen concentration in the measurement-object gas, by instantly changing the control voltage (voltage V0) for the main pump cell 21 in the first internal cavity 20 to, for example, about 200 mV to about 700 mV. Generally, an oxygen pump voltage (pump voltage Vp0) applied between the intracavity oxygen pump electrode (inner main pump electrode 22) and the extracavity oxygen pump electrode (outer pump electrode 23) in the oxygen measurement mode is to be lower than an oxygen pump voltage (pump voltage Vp0) applied between the intracavity oxygen pump electrode (inner main pump electrode 22) and the extracavity oxygen pump electrode (outer pump electrode 23) in the normal measurement mode.

### Variation

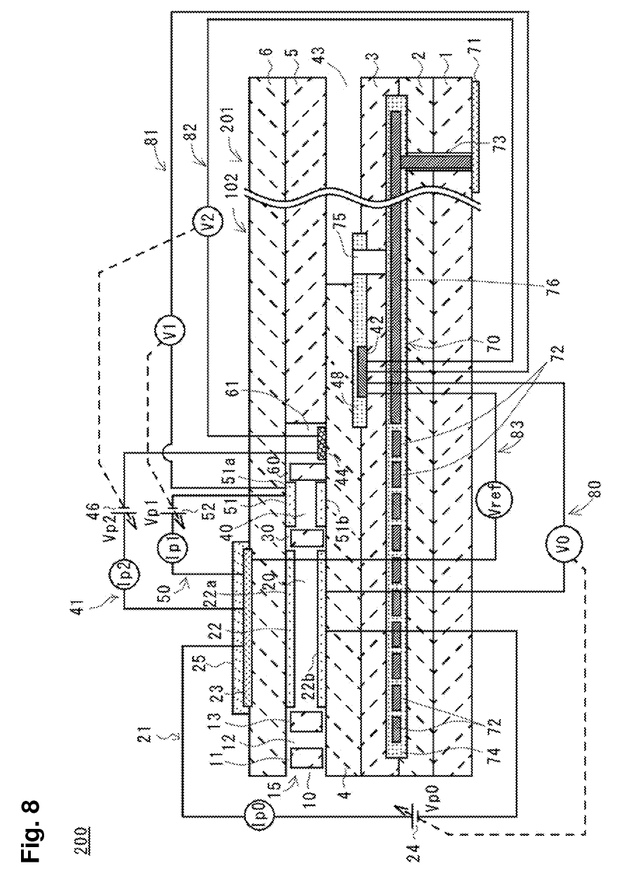

In the above-described embodiment, in the oxygen measurement mode, oxygen is pumped out from the first internal cavity 20, and based on the pump current Ip0 flowing at the time, the oxygen concentration in the oxygen measurement mode is measured. However, the present invention is not limited thereto. FIG. 8 is a vertical sectional schematic view in the longitudinal direction of a sensor element 201, showing one example of a general configuration of the gas sensor 200 including the sensor element 201 of a variation. In FIG. 8, the same member as in FIG. 1 is denoted by the same sign.

In the gas sensor 200, the sensor element 201 further includes a diffusion resister arranged around the extracavity oxygen pump electrode (outer pump electrode 23), in addition to the configuration of the sensor element 101. In the sensor element 201, a porous layer 25 functions as the diffusion resister. The diffusion resister may be in such a form that a desired diffusion resistance is created to the measurement-object gas that reaches the outer pump electrode 23, but the form is not limited to the porous layer 25. For example, a space whose walls are formed of a dense body may be arranged around the outer pump electrode 23, and a hole for introducing the measurement-object gas exists on a part of the wall.

In the oxygen measurement mode, a pump control part (not shown) of the gas sensor 200 is configured to apply a predetermined oxygen pump voltage (pump voltage Vp0) at which neither the water vapor nor the carbon dioxide is decomposed on the extracavity oxygen pump electrode (the outer pump electrode 23) between the intracavity oxygen pump electrode (the inner main pump electrode 22) and the extracavity oxygen pump electrode (the outer pump electrode 23) to pump oxygen into the first internal cavity 20. The pump control part of the gas sensor 200 may perform, for example, the following feedback control based on an electromotive force (a voltage V0) generated in the oxygen-partial-pressure detection sensor cell 80 for main pump control to pump oxygen into the first internal cavity 20. Specifically, in the gas sensor 200, control of the oxygen measurement mode is performed in the following manner.

In the oxygen measurement mode, the pump control part of the gas sensor 200 performs feedback control of the pump voltage Vp0 of the variable power supply 24 in the main pump cell 21 so that the electromotive force (the voltage V0) generated between the intracavity oxygen pump electrode (the inner main pump electrode 22) and the reference electrode 42 is at a constant value (referred to as a set value V0b_{SET}). The set value V0b_{SET} is set as a control voltage in the oxygen measurement mode with respect to the main pump cell 21. The set value V0b_{SET} may be set as a value such that oxygen is pumped into the first internal cavity 20 to remove all of, or substantially all of oxygen in the atmosphere in the vicinity of the outer pump electrode 23 covered by the porous layer 25 that is the diffusion resister. For example, the set value V0b_{SET} may be set as a value such that the pump current Ip0 is to be a limiting current in the direction of pumping oxygen into the first internal cavity 20. The set value V0b_{SET} may vary depending on the intended use of the gas sensor 200, the configuration of the sensor element 201 and the like, and the set value V0b_{SET} may be, for example, about -700 (minus 700) mV or more and about -200 (minus 200) mV or less.

Alternatively, in the oxygen measurement mode, the pump control part of the gas sensor 200 may perform, for example, the following feedback control based on an electromotive force (a voltage Vref) generated in the sensor cell 83 to pump oxygen into the first internal cavity 20. Specifically, in the gas sensor 200, control of the oxygen measurement mode may be performed in the following manner.

In the oxygen measurement mode, the pump control part of the gas sensor 200 may perform feedback control of the pump voltage Vp0 of the variable power supply 24 in the main pump cell 21 so that the electromotive force (the voltage Vref) generated between the extracavity oxygen pump electrode (the outer pump electrode 23) and the reference electrode 42 is at a constant value (referred to as a set value Vref_{SET}). The set value Vref_{SET} is set as a control voltage in the oxygen measurement mode with respect to the main pump cell 21. The set value Vref_{SET} may be set as a value such that oxygen is pumped into the first internal cavity 20 to remove all of, or substantially all of oxygen in the atmosphere in the vicinity of the outer pump electrode 23 covered by the porous layer 25 that is the diffusion resister. For example, the set value Vref_{SET} may be set as a value such that the pump current Ip0 is to be a limiting current in the direction of pumping oxygen into the first internal cavity 20. The set value Vref_{SET} may vary depending on the intended use of the gas sensor 200, the configuration of the sensor element 201 and the like, and the set value Vref_{SET} may be, for example, about 200 mV or more and about 700 mV or less.

The gas sensor 100 and the gas sensor 200 has been described above as examples of the embodiment according to the present invention, but the present invention is not limited thereto. The present invention may include a gas sensor having any structure as long as the object of the present invention can be achieved, that is, a gas sensor that can accurately measure a water vapor concentration and a carbon dioxide concentration in a measurement-object gas regardless of an oxygen concentration in the measurement-object gas is provided.

In the gas sensor 100 of the above-described embodiment, an example where a deviation amount from an ideal value of the pump current Ip1 is used as a correction value is shown in FIG. 5. However, the correction value is not limited thereto. For example, a ratio of the ideal value to an actual pump current Ip1 may be used as the correction value. In this case, a corrected pump current Ip1 may be calculated by multiplying the pump current Ip1 by the correction value. Further, an example where a deviation amount from an ideal value of the pump current Ip2 is used as a correction value is shown in FIG. 6. However, the correction value is not limited thereto. For example, a ratio of the ideal value to an actual pump current Ip2 may be used as the correction value. In this case, a corrected pump current Ip2 may be calculated by multiplying the pump current Ip2 by the correction value.

In the gas sensor 100 of the above-described embodiment, the concentration calculating part 93 calculates a correction value with respect to each of the pump current Ip1 and the pump current Ip2 based on the oxygen concentration in the oxygen measurement mode, corrects each of the pump current Ip1 and the pump current Ip2 by the correction value to calculate each of a corrected pump current Ip1 and a corrected pump current Ip2, and calculates the water vapor concentration and the carbon dioxide concentration based on the corrected pump current Ip1 and the corrected pump current Ip2, respectively. However, the present invention is not limited thereto. The concentration calculating part 93 may calculate a tentative water vapor concentration and a tentative carbon dioxide concentration from the pump current Ip1 and the pump current Ip2 acquired in the normal measurement mode, respectively, and then, may correct the tentative water vapor concentration and the tentative carbon dioxide concentration based on the oxygen concentration in the oxygen measurement mode to obtain the water vapor concentration and the carbon dioxide concentration.

In the gas sensor 100 of the above-described embodiment, the concentration calculating part 93 is configured to output the water vapor concentration and the carbon dioxide concentration as measurement values of the gas sensor 100. However, the present invention is not limited thereto. The concentration calculating part 93 may be configured to output either the water vapor concentration or the carbon dioxide concentration as the measurement value of the gas sensor 100. Further, the concentration calculating part 93 may be configured to output the calculated oxygen concentration in the oxygen measurement mode as a measurement value of the gas sensor 100.

In the gas sensor 100 of the above-described embodiment, the concentration calculating part 93 calculates a correction value with respect to each of the pump current Ip1 and the pump current Ip2 based on the oxygen concentration calculated from the current (pump current Ip0) flowing through the main pump cell 21 in the oxygen measurement mode. However, the present invention is not limited thereto. For example, the concentration calculating part 93 may calculate the correction value with respect to each of the pump current Ip1 the pump current Ip2 based on the pump current Ip0 directly, without the calculation of the oxygen concentration in the measurement-object gas.

In the gas sensor 100 of the above embodiment, the inner main pump electrode 22 is composed of the ceiling electrode portion 22a formed on the ceiling surface of the first internal cavity 20, the bottom electrode portion 22b formed on the bottom surface of the first internal cavity 20, and the lateral electrode portions formed on the lateral wall surfaces of the first internal cavity 20 so as to connect the ceiling electrode portion 22a and the bottom electrode portion 22b. However, the inner main pump electrode 22 is not limited thereto. For example, the inner main pump electrode 22 may be formed only on the ceiling surface of the first internal cavity 20. Alternatively, the inner main pump electrode 22 may be formed only on the bottom surface of the first internal cavity 20. For example, when the inner main pump electrode 22 has the ceiling electrode portion 22a and the bottom electrode portion 22b, the ceiling electrode portion 22a and the bottom electrode portion 22b may be the same in size, or the size of the ceiling electrode portion 22a and the bottom electrode portion 22b may be different from each other. The same is true for the first measurement electrode 51. Further, the second measurement electrode 44 is formed on the bottom surface of the third internal cavity 61. However, the second measurement electrode 44 is not limited thereto. The second measurement electrode 44 may be formed on the ceiling surface of the third internal cavity 61. Alternatively, the second measurement electrode 44 may be in a tunnel-like structure similar to the inner main pump electrode 22.

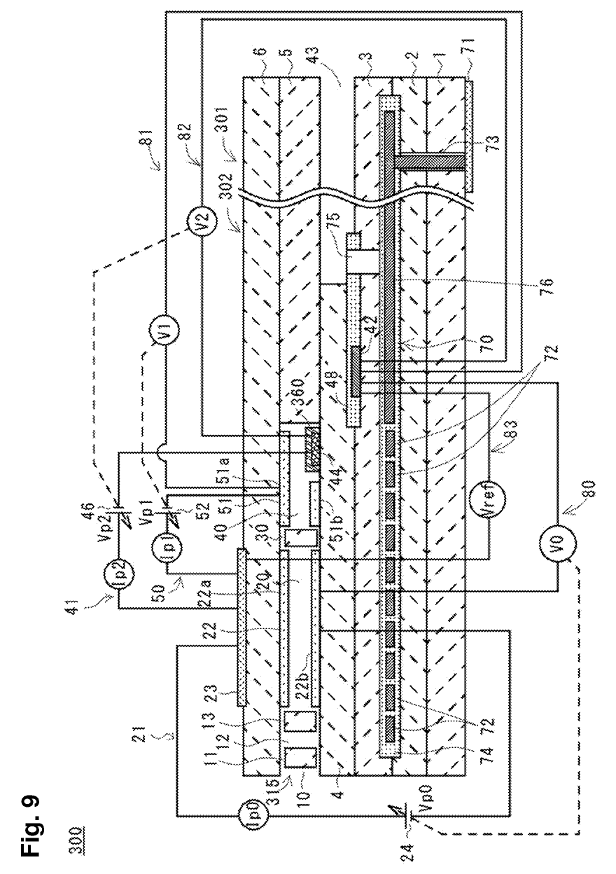

As shown in FIG. 1, in the gas sensor 100 of the above-described embodiment, the sensor element 101 has a structure in which the first measurement electrode 51 is disposed in the second internal cavity 40, and the second measurement electrode 44 is disposed in the third internal cavity 61. However, the structure of the sensor element is not limited thereto. For example, the sensor element may have a structure in which the third internal cavity 61 is not provided, and the first measurement electrode 51 and the second measurement electrode 44 are disposed in the second internal cavity 40 as shown in FIG. 9. FIG. 9 is a vertical sectional schematic view in the longitudinal direction, showing one example of a schematic configuration of a gas sensor 300 including a sensor element 301. In FIG. 9, the same member as in FIG. 1 is denoted by the same sign.

In the sensor element 301, a second internal cavity 40 of a measurement-object gas flow cavity 315 is formed to extend further to the rear end side than a second internal cavity 40 in the sensor element 101 in the longitudinal direction of the sensor element. The second measurement electrode 44 is disposed at a position opposite to the second diffusion-rate limiting path 30 with respect to the first measurement electrode 51, schematically, at a position farther from the second diffusion-rate limiting path 30 than the first measurement electrode 51, in the second internal cavity 40. The second measurement electrode 44 is disposed on the upper surface of the first solid electrolyte layer 4 in the second internal cavity 40. The second measurement electrode 44 is covered with a third diffusion-rate limiting layer 360 that functions as a third diffusion-rate limiting path. The third diffusion-rate limiting layer 360 is a porous alumina layer, which is a part that creates a predetermined diffusion resistance to the measurement-object gas expected to come into contact with the second measurement electrode 44 in the second internal cavity 40. The third diffusion-rate limiting layer 360 also functions as an electrode protecting layer that protects the second measurement electrode 44 from, for example, particle adhesion. The porous third diffusion-rate limiting layer 360 serves a similar role as the third diffusion-rate limiting path 60 in the sensor element 101. Therefore, an oxygen concentration, a water vapor concentration, and a carbon dioxide concentration in a measurement-object gas in an objective space can be measured in the same manner as in the case of the gas sensor 100 of the above-described embodiment.

In the gas sensor 100 of the above-described embodiment, the outer pump electrode 23 has three functions as an extracavity oxygen pump electrode in the main pump cell 21, an extracavity first measurement electrode in the first measurement pump cell 50, and an extracavity second measurement electrode in the second measurement pump cell 41. However, the outer pump electrode 23 is not limited thereto. For example, the extracavity oxygen pump electrode, the extracavity first measurement electrode, and the extracavity second measurement electrode may be formed as different electrodes. For example, any one or more of the extracavity oxygen pump electrode, the extracavity first measurement electrode, and the extracavity second measurement electrode may be provided on the outer surface of the base part 102 separately from the outer pump electrode 23 so as to be in contact with a measurement-object gas. Alternatively, the reference electrode 42 may also serve as any one or more of the extracavity oxygen pump electrode, the extracavity first measurement electrode, and the extracavity second measurement electrode.

### Method for producing gas sensor

Next, one example of a method for producing the gas sensor as described above is described. A plurality of unfired sheet moldings (so-called green sheets) containing an oxygen-ion-conductive solid electrolyte such as zirconia (ZrO₂) as a ceramic component are subjected to a predetermined processing and printing of circuit pattern, and then the plurality of sheets are laminated, and the laminate was cut, and then fired. Thus the sensor element 101 can be manufactured. Then, the manufactured sensor element may be incorporated into the gas sensor.

Hereinafter, description is made while taking the case of manufacturing the sensor element 101 composed of six layers shown in FIG. 1 as an example.

First, six green sheets containing an oxygen-ion-conductive solid electrolyte such as zirconia (ZrO₂) as a ceramic component are prepared. For manufacturing of the green sheets, a known molding method can be used. The six green sheets may all have the same thickness, or the thickness differs depending on the layer to be formed. In each of the six green sheets, sheet holes or the like for use in positioning at the time of printing or stacking are formed in advance by a known method such as a punching process with a punching apparatus to prepare a blank sheet. In the blank sheet for use as the spacer layer 5, penetrating parts such as internal cavities are also formed in the same manner. Also in the remaining layers, necessary penetrating parts are formed in advance.

The blank sheets for use as six layers, namely, the first substrate layer 1, the second substrate layer 2, the third substrate layer 3, the first solid electrolyte layer 4, the spacer layer 5, and the second solid electrolyte layer 6 are subjected to printing of various patterns required for respective layers and drying treatment. For printing of a pattern, a known screen printing technique can be used. Also as the drying treatment, a known drying means can be used.

After completing the printing and drying of diverse patterns for each of the six blank sheets by repeating these steps, contact bonding treatment of stacking the six printed blank sheets in a predetermined order while positioning with the sheet holes and the like, and contact bonding at a predetermined temperature and pressure condition to give a laminate is conducted. The contact bonding treatment is conducted by heating and pressurizing with a known laminator such as a hydraulic press. While the temperature, the pressure and the time of heating and pressurizing depend on the laminator being used, they may be appropriately determined to achieve excellent lamination.

The obtained laminate includes a plurality of sensor elements 101. The laminate is cut into units of the sensor element 101. The cut laminate is fired at a predetermined firing temperature to obtain the sensor element 101. That is, the sensor element 101 is obtained by integral firing of the solid electrolyte layers and the electrodes. The firing temperature may be such a temperature that the solid electrolyte forming the base part 102 of the sensor element 101 is sintered to become a dense product, and an electrode or the like maintains desired porosity. The firing is conducted, for example, at a firing temperature of about 1200°C or more and 1500°C or less.

The obtained sensor element 101 is incorporated into the gas sensor 100 in such a form that the front end part of the sensor element 101 comes into contact with the measurement-object gas, and the rear end part of the sensor element 101 comes into contact with the reference gas.

As described above, according to the present invention, the pump current Ip1 as the water vapor detecting current and the pump current Ip2 as the carbon dioxide detecting current can be corrected based on an oxygen concentration in a measurement-object gas. Therefore, it is possible to provide a gas sensor that can accurately measure a water vapor concentration and a carbon dioxide concentration in a measurement-object gas regardless of an oxygen concentration in the measurement-object gas.

### EXPLANATION OF REFERENCE SIGNS IN THE DRAWINGS

1: first substrate layer; 2: second substrate layer; 3: third substrate layer; 4: first solid electrolyte layer; 5: spacer layer; 6: second solid electrolyte layer; 10: gas inlet; 11: first diffusion-rate limiting path; 12: buffer space; 13: fourth diffusion-rate limiting path; 15, 315: measurement-object gas flow cavity; 20: first internal cavity; 21: main pump cell; 22: inner main pump electrode; 22a: ceiling electrode portion (of the inner main pump electrode); 22b: bottom electrode portion (of the inner main pump electrode); 23: outer pump electrode; 24: variable power supply (of the main pump cell); 25: porous layer; 30: second diffusion-rate limiting path; 40: second internal cavity; 41: second measurement pump cell; 42: reference electrode; 43: reference gas introduction space; 44: second measurement electrode; 46: variable power supply (of the second measurement pump cell); 48: air introduction layer; 50: first measurement pump cell; 51: first measurement electrode; 51a: ceiling electrode portion (of the first measurement pump cell); 51b: bottom electrode portion (of the first measurement pump cell); 52: variable power supply (of the first measurement pump cell); 60: third diffusion-rate limiting path; 61: third internal cavity; 70: heater part; 71: heater electrode; 72: heater; 73 through hole; 74 heater insulating layer; 75: pressure relief vent; 76: heater lead; 80: oxygen-partial-pressure detection sensor cell for main pump control; 81: oxygen-partial-pressure detection sensor cell for first measurement pump control; 82: oxygen-partial-pressure detection sensor cell for second measurement pump control; 83: sensor cell; 90: control unit; 91: control part; 92: pump control part; 93: concentration calculating part; 100, 200, 300: gas sensor; 101, 201, 301: sensor element; and 102, 302: base part.

What is claimed is:

1. A gas sensor for detecting water vapor and carbon dioxide in a measurement-object gas, the gas sensor comprising a sensor element and a control unit for controlling the sensor element, wherein
the sensor element comprises:
a base part in an elongated plate shape, including an oxygen-ion-conductive solid electrolyte layer;
a measurement-object gas flow cavity having a gas inlet that opens on a surface of the base part, a first internal cavity that communicates with the gas inlet via a first diffusion-rate limiting path, and a second internal cavity that communicates with the first internal cavity via a second diffusion-rate limiting path;
an oxygen pump cell including: an intracavity oxygen pump electrode disposed in the first internal cavity of the measurement-object gas flow cavity; and an extracavity oxygen pump electrode disposed at a position different from the measurement-object gas flow cavity on the base part and corresponding to the intracavity oxygen pump electrode;
a first measurement pump cell including: an intracavity first measurement electrode disposed in the second internal cavity of the measurement-object gas flow cavity; and an extracavity first measurement electrode disposed at a position different from the measurement-object gas flow cavity on the base part and corresponding to the intracavity first measurement electrode;
a second measurement pump cell including: an intracavity second measurement electrode disposed at a position opposite to the second diffusion-rate limiting path with respect to the intracavity first measurement electrode in the second internal cavity, or in a third internal cavity that communicates with the second internal cavity via a third diffusion-rate limiting path of the measurement-object gas flow cavity; and an extracavity second measurement electrode disposed at a position different from the measurement-object gas flow cavity on the base part and corresponding to the intracavity second measurement electrode;
a reference gas chamber formed inside the base part, and being separated from the measurement-object gas flow cavity; and
a reference electrode disposed in the reference gas chamber; and
the control unit comprises:
a pump control part for controlling operation of the oxygen pump cell, the first measurement pump cell and the second measurement pump cell, and
a concentration calculating part for calculating a concentration of each of water vapor and carbon dioxide in a measurement-object gas, wherein
the pump control part has an oxygen measurement mode and a normal measurement mode;
in the oxygen measurement mode, the pump control part controls the operation of the oxygen pump cell, and
in the normal measurement mode,
the pump control part applies a predetermined oxygen pump voltage between the intracavity oxygen pump electrode and the extracavity oxygen pump electrode of the oxygen pump cell to pump out oxygen from the first internal cavity and decompose the water vapor and the carbon dioxide in the measurement-object gas in the first internal cavity;
the pump control part applies a predetermined first measurement pump voltage between the intracavity first measurement electrode and the extracavity first measurement electrode of the first measurement pump cell to pump oxygen into the second internal cavity and selectively oxidize, in the second internal cavity, hydrogen generated by decomposition of the water vapor; and
the pump control part applies a predetermined second measurement pump voltage between the intracavity second measurement electrode and the extracavity second measurement electrode of the second measurement pump cell to pump oxygen into near a surface of the intracavity second measurement electrode and selectively oxidize, near the surface of the intracavity second measurement electrode, carbon monoxide generated by decomposition of the carbon dioxide; and
the concentration calculating part calculates an oxygen concentration in the oxygen measurement mode;
the concentration calculating part calculates a water vapor concentration in the measurement-object gas based on a current flowing through the first measurement pump cell in the normal measurement mode and the oxygen concentration in the oxygen measurement mode; and
the concentration calculating part calculates a carbon dioxide concentration in the measurement-object gas based on a current flowing through the second measurement pump cell in the normal measurement mode and the oxygen concentration in the oxygen measurement mode.

2. The gas sensor according to claim 1, wherein
in the oxygen measurement mode, the pump control part applies a predetermined oxygen pump voltage at which neither the water vapor nor the carbon dioxide is decomposed on the intracavity oxygen pump electrode between the intracavity oxygen pump electrode and the extracavity oxygen pump electrode to pump out oxygen from the first internal cavity; and
the concentration calculating part calculates the oxygen concentration in the oxygen measurement mode based on a current flowing though the oxygen pump cell in the oxygen measurement mode.

3. The gas sensor according to claim 2, wherein
in the oxygen measurement mode, the pump control part applies the predetermined oxygen pump voltage at which neither the water vapor nor the carbon dioxide is decomposed on the intracavity oxygen pump electrode between the intracavity oxygen pump electrode and the extracavity oxygen pump electrode based on a voltage between the intracavity oxygen pump electrode and the reference electrode to pump out oxygen from the first internal cavity.

4. The gas sensor according to claim 2, wherein the predetermined oxygen pump voltage applied between the intracavity oxygen pump electrode and the extracavity oxygen pump electrode in the oxygen measurement mode is lower than the predetermined oxygen pump voltage applied between the intracavity oxygen pump electrode and the extracavity oxygen pump electrode in the normal measurement mode.

5. The gas sensor according to claim 3, wherein the voltage between the intracavity oxygen pump electrode and the reference electrode is controlled at 200 mV or more and 700 mV or less in the oxygen measurement mode.

6. The gas sensor according to claim 1, wherein
the sensor element further comprises a diffusion resister arranged around the extracavity oxygen pump electrode;
in the oxygen measurement mode, the pump control part applies a predetermined oxygen pump voltage at which neither the water vapor nor the carbon dioxide is decomposed on the extracavity oxygen pump electrode between the intracavity oxygen pump electrode and the extracavity oxygen pump electrode to pump oxygen into the first internal cavity; and
the concentration calculating part calculates the oxygen concentration in the oxygen measurement mode based on a current flowing though the oxygen pump cell in the oxygen measurement mode.

7. The gas sensor according to claim 6, wherein
in the oxygen measurement mode, the pump control part applies the predetermined oxygen pump voltage at which neither the water vapor nor the carbon dioxide is decomposed on the extracavity oxygen pump electrode between the intracavity oxygen pump electrode and the extracavity oxygen pump electrode based on a voltage between the intracavity oxygen pump electrode and the reference electrode to pump oxygen into the first internal cavity.

8. The gas sensor according to claim 7, wherein the voltage between the intracavity oxygen pump electrode and the reference electrode is controlled at -700 mV or more and -200 mV or less in the oxygen measurement mode.

9. The gas sensor according to claim 6, wherein
in the oxygen measurement mode, the pump control part applies the predetermined oxygen pump voltage at which neither the water vapor nor the carbon dioxide is decomposed on the extracavity oxygen pump electrode between the intracavity oxygen pump electrode and the extracavity oxygen pump electrode based on a voltage between the extracavity oxygen pump electrode and the reference electrode to pump oxygen into the first internal cavity.

10. The gas sensor according to claim 9, wherein the voltage between the extracavity oxygen pump electrode and the reference electrode is controlled at 200 mV or more and 700 mV or less in the oxygen measurement mode.

11. The gas sensor according to claim 6, wherein an absolute value of the predetermined oxygen pump voltage applied between the intracavity oxygen pump electrode and the extracavity oxygen pump electrode in the oxygen measurement mode is lower than an absolute value of the predetermined oxygen pump voltage applied between the intracavity oxygen pump electrode and the extracavity oxygen pump electrode in the normal measurement mode.

12. The gas sensor according to claim 6, wherein the diffusion resister is a porous layer covering the extracavity oxygen pump electrode.

13. The gas sensor according to claim 1, wherein
the concentration calculating part corrects the current flowing through the first measurement pump cell in the normal measurement mode based on the oxygen concentration in the oxygen measurement mode to calculate a corrected current, and calculates the water vapor concentration in the measurement-object gas based on the corrected current; and
the concentration calculating part corrects the current flowing through the second measurement pump cell in the normal measurement mode based on the oxygen concentration in the oxygen measurement mode to calculate a corrected current, and calculates the carbon dioxide concentration in the measurement-object gas based on the corrected current.

14. The gas sensor according to claim 1, wherein in the normal measurement mode,
the pump control part applies the predetermined oxygen pump voltage between the intracavity oxygen pump electrode and the extracavity oxygen pump electrode of the oxygen pump cell based on a voltage between the intracavity oxygen pump electrode and the reference electrode to pump out oxygen from the first internal cavity and decompose the water vapor and the carbon dioxide in the measurement-object gas in the first internal cavity;
the pump control part applies the predetermined first measurement pump voltage between the intracavity first measurement electrode and the extracavity first measurement electrode of the first measurement pump cell based on a voltage between the intracavity first measurement electrode and the reference electrode to pump oxygen into the second internal cavity and selectively oxidize, in the second internal cavity, the hydrogen generated by the decomposition of the water vapor; and
the pump control part applies the predetermined second measurement pump voltage between the intracavity second measurement electrode and the extracavity second measurement electrode of the second measurement pump cell based on a voltage between the intracavity second measurement electrode and the reference electrode to pump oxygen into near the surface of the intracavity second measurement electrode and selectively oxidize, near the surface of the intracavity second measurement electrode, the carbon monoxide generated by the decomposition of the carbon dioxide.

15. The gas sensor according to claim 1, wherein the pump control part repeatedly performs the oxygen measurement mode and the normal measurement mode.

16. The gas sensor according to claim 1, wherein
a duration time of the oxygen measurement mode is 0.1 second or more and 3 seconds or less, and
a duration time of the normal measurement mode is 1 second or more and 20 seconds or less.

17. A control method of a gas sensor for detecting water vapor and carbon dioxide in a measurement-object gas, the gas sensor being the gas sensor according to claim 1, and
the control method comprising:
an oxygen concentration measuring step of calculating an oxygen concentration in the oxygen measurement mode;
a water vapor concentration measuring step of calculating a water vapor concentration in the measurement-object gas based on a current flowing through the first measurement pump cell in the normal measurement mode and the oxygen concentration in the oxygen measurement mode just before the normal measurement mode; and
a carbon dioxide concentration measuring step of calculating a carbon dioxide concentration in the measurement-object gas based on a current flowing through the second measurement pump cell in the normal measurement mode and the oxygen concentration in the oxygen measurement mode just before the normal measurement mode.
